(19) United States
(12) Patent Application Publication
(10) Pub. No.: US 2016/0223536 A1
(43) Pub. Date: Aug. 4, 2016

(54) Improved Lateral Flow Assays

(71) Applicant: SONG DIAGNOSTIC RESEARCH LLC, Menlo Park, CA (US)

(72) Inventors: Martin D Johnson, Woodside, CA (US); Linda G Lee, Palo Alto, CA (US); Eric S Nordman, Palo Alto, CA (US); Mark F Oldham, Emerald Hills, CA (US)

(73) Assignee: Song Diagnostic Research LLC, Menio Park, CA (US)

(21) Appl. No.: 15/025,274

(22) PCT Filed: Oct. 9, 2014
(86) PCT No.: PCT/US2014/059979

§ 371 Date: Mar. 26, 2016

Related U.S. Application Data
(60) Provisional application No. 62/057,214, filed on Sep. 29, 2014.
(60) Provisional application No. 61/961,428, filed on Oct. 15, 2013.

(51) Int. Cl. G01N 33/558; G01N 33/68; G01N 21/64; G01N 33/543; G01N 33/552

(52) U.S. Cl. CPC G01N 33/558; G01N 33/54393; G01N 33/552; G01N 33/54366; G01N 21/6428; G01N 21/645; G01N 33/6893; G01N 33/54306; G01N 2021/6439; G01N 2201/062; G01N 2333/161; G01N 2800/52; G01N 2800/26

(57) ABSTRACT

Lateral flow test strips, systems, and methods are provided for measuring the presence and levels of analytes in samples in which the analyte may be complexed, for example within an analyte-antibody complex. Test strips are provided that can decomplex the analyte from the analyte-antibody complex during the lateral flow assay, resulting in high quality assays without the need for a decomplexation pre-treatment step. Various systems and methods for improving the performance of lateral flow assays are described, which include minimization of the Prozone effect, improved dynamic range, improving sensitivity by disrupting complexation of target antigens. The resulting lateral flow system has improved sensitivity and improved dynamic range, and may utilize fluorescence. The illumination system utilizes an LED, plastic lenses and plastic and colored glass filters for the excitation and emission light.

### CROSS REFERENCE TO RELATED APPLICATIONS

This application claims priority to Provisional Patent Applications 61/961,428, filed Oct. 10, 2013 and 62/057,214, filed Sep. 29, 2014, which are incorporated herein by reference in their entirety for all purposes.

### BACKGROUND OF THE INVENTION

Lateral flow assays (LFAs) are used for many diagnostic tests due to their low cost and simple operation. Most LFA tests use colloidal gold reporters with visual readout. As such they are not quantitative and often have inadequate sensitivity. Even when the colloidal gold LFA is scanned, the limited linear response range of the absorbance and or reflectance measurement leads to a small dynamic range, even when a curve-fitting algorithm is utilized to compensate for non-linearities in the signal response curve.

Some LFAs use fluorescence detection, but the detection systems are expensive. Especially in resource-limited areas such as rural clinics in developing nations, there exists a need for low cost stand alone devices that improve point-of-care diagnosis. Improvements may come in the form of improved sensitivity, quantitative results, increased dynamic range and ease of use.

Lateral flow assay technology is used for the detection of proteins, viral antigens and small molecules, and enables rapid point-of-care diagnostics of infectious diseases such as malaria, syphilis, dengue, and HIV, as well as cardiac markers such as troponin, and cancer biomarkers such as prostate specific antigen (PSA). The most common format utilizes a sandwich immunoassay: two antibodies are ultimately bound to an analyte in a sandwich fashion. One antibody is initially bound, typically non-covalently, in a horizontal stripe on a narrow strip of nitrocellulose. The remaining nitrocellulose surface may be blocked with protein(s) to prevent nonspecific adherence of analyte and or other proteins, and the analyte and a second, labeled antibody are allowed to flow up the nitrocellulose. A “sandwich” of the analyte and the two antibodies forms on the stripe and appears as a visible, reddish line. Typically, an absorbent pad containing the labeled antibody is used to deliver the reagent, and a control line comprising antibody specific to the Fragment crystallizable (Fc) region of the labeled antibody is located upstream of the test line.

The most common label or reporter entity is colloidal gold. Antibodies can be noncovalently or covalently bound or attached to gold, and visual detection of the stripe can be simple and robust when the assay is performed with analyte quantities within the dynamic range of the assay. Gold is stable under exposure to heat and light; degradation is limited primarily by the stability of the protein(s). Disadvantages include a very limited quantitative dynamic range and a limit of detection which is often inadequate even with expensive reader systems.

An advantage of fluorescence over absorbance systems is the dark and uniform background that is achieved by efficient blocking of the excitation light. Fluorescence detection also provides a wide dynamic range since the light emitted is proportional to the concentration while the amount of light reflected after absorption is a nonlinear function of concentration. Generally, fluorescence systems tend to be expensive due to the expensive light sources required to illuminate the fluorescent reporters, the interference filters and detection systems required to process and capture the emitted light, and the data processing required to produce the result. Several reports have described the use of fluorescence in lateral flow systems, but their results do not show a sufficient advantage of using fluorescence instead of gold in either sensitivity or dynamic range that would justify the extra cost and complexity.

Various diseases require measurements of targets which may normally be inaccessible due to complexation, which may be complexes of antibodies and RNA or antibodies and proteins, as occurs with HIV P24 RNA assays and with Dengue fever NS1 protein assays. The binding of the antibody may render the target unavailable, as the target area may be the same for a capture or label antibody and the antibody with which the target is complexed. It may thus be desirable to disrupt or otherwise cause disassociation of complexes of target moieties.

Herein is described lateral flow test strips, systems and methods for improved detection and quantitation of levels of analytes in samples where the analyte may be complexed, for example by patient antibodies in a sample. We describe an inexpensive reader system using an LED light source(s) and readily available plastic and colored glass filters. The system described herein may include a phone application that would enable on-phone data processing with the data processor on the phone, and reporting, thus providing all computer functions on the mobile device. The system may be utilized with various fluorescent reporters for use in lateral flow assays.

### BRIEF DESCRIPTION OF THE INVENTION

In some aspects the invention provides a lateral flow test strip for detecting analyte levels in a sample comprising: a sample application region; a decomplexation region for dissociating analyte-antibody complexes in the sample; a conjugate region comprising a detection antibody that selectively associates with the analyte; a flow region; and a test line comprising immobilized test antibody.

In some cases the lateral flow test strip of further comprises a neutralization region comprising neutralizing agents that neutralize the decomplexation reagent. In some cases the lateral flow test strip further comprises an elution reagent application region on the strip upstream of the sample application region. In some cases the strip is configured such that the elution reagent combined with the sample is added to the sample application region of the strip. In some cases the decomplexation region comprises an acidification reagent that lowers the pH of the sample as the sample passes through the decomplexation region. In some cases the decomplexation region comprises an acidification reagent that lowers the pH of the sample as the sample passes through the decomplexation region, and wherein the neutralizing reagent comprises a base that raises the pH of the sample as it passes through the neutralization region. In some cases the acidification reagent brings the pH of the sample to less than about 5. In some cases the acidification reagent brings the pH of the sample to less than about 4. In some cases the acidification reagent brings the pH of the sample to less than about 3.

In some cases the acidification reagent comprises citric acid, glycine-HCl, or tartaric acid. In some cases the acidification reagent comprises a polymeric cation exchanger in the acid form. In some cases the acidification reagent comprises a carboxylic acid, a sulfonic acid, a phosphoric acid or a phosphonic acid.

In some cases the decomplexation region comprises a detergent. In some cases the detergent comprises sodium dodecyl sulfonate.

In some cases the decomplexation region raises the salt concentration in the sample for decomplexation. In some cases the salt comprises lithium chloride, magnesium chloride, or sodium thiocyanate.

In some cases the decomplexation region provides an organic solvent into the sample for decomplexation. In some cases the organic solvent comprises ethylene glycol.

In some cases the decomplexation region comprises a chaotropic agent. In some cases the chaotropic agent comprises urea or guanidine-HCl.

In some cases the decomplexation region is a region that is heated. In some cases heating is provided by a compound that gives off heat when it comes in contact with the elution reagent. In some cases the heating is provided by an electric heater.

In some cases the detection antibody comprises a fluorescent label. In some cases the decomplexation region and the sample application region are coextensive. In some cases the neutralization region is coextensive with the conjugate region.

In some aspects the invention provides a lateral flow test strip for detecting analyte levels in a sample comprising: a sample application region; a decomplexation region comprising a dissociating reagent for dissociating analyte-antibody complexes in the sample, a conjugate region comprising a detection antibody that selectively associates with the analyte; wherein the sample is mixed with an elution reagent that comprises components which result in the neutralization of the dissociating reagent before it reaches the conjugate region; a flow region; and a test line comprising immobilized test antibody.

In some aspects the invention provides a lateral flow test strip for detecting analyte levels in a sample comprising: a sample application region; a decomplexation region comprising a heated region which provides heat to dissociate analyte-antibody complexes in the sample; a conjugate region comprising a detection antibody that selectively associates with the analyte; a flow region; and a test line comprising immobilized test antibody.

In some aspects, the invention provides a method for detecting an analyte, which analyte may comprise analyte-antibody complexes, in a sample comprising: providing a test strip comprising; a sample application region; a decomplexation region for dissociating analyte-antibody complexes in the sample; a conjugate region comprising a detection antibody that selectively associates with the analyte; a flow region; and a test line comprising immobilized test antibody, and applying a sample to sample application region; whereby the sample flows down the strip such that when the sample is in the decomplexation region at least some of the analyte-antibody complex is dissociated, and whereby the sample passes through the test line whereby the presence of analyte is detected by complexation with the immobilized test antibody. In some cases the method provides for measuring the level of analyte in the sample. In some cases the analyte comprises p24 analyte. In some cases the level of p24 analyte in the sample is measured.

In some aspects, the invention provides a method of determining the level of p24 in a patient in order to manage HIV therapy comprising; obtaining a sample from a patient; applying the sample to a lateral flow test strip described herein; determining the level of p24 in the sample; using the determined level of p24 to manage the care of the HIV patient. In some cases the sample comprises whole blood from the patient. In some cases the sample comprises serum from the patient. In some cases the p24 level is determined using a fluorescently labeled detection antibody.

In some aspects the invention provides a dual flow test strip for detecting analyte levels in a sample in which the analyte may be complexed comprising: a sample application region; wherein the strip comprises, downstream of the sample application region, a first lane and a second lane, wherein the first lane comprises; a decomplexation region for dissociating analyte-antibody complexes in the sample; a first lane flow region; and a first lane test line comprising immobilized test antibody; wherein the second lane does not have a decomplexation region and comprises; a second lane flow region; and a second lane test line comprising immobilized test antibody.

In some aspects, the invention provides a method for measuring both decomplexed and complexed analyte levels in a sample comprising: adding a sample containing analyte that may be complexed to dual flow test strip, the test strip comprising: a sample application region; wherein the strip comprises, after the sample application region, a first lane and a second lane, wherein the first lane comprises; a decomplexation region for dissociating analyte-antibody complexes in the sample; a first lane conjugate region comprising a detection antibody that selectively associates with the analyte a first lane flow region; and a first lane test line comprising immobilized test antibody wherein the second lane does not have a decomplexation region and comprises; a second lane conjugate region comprising a detection antibody that selectively associates with the analyte a second lane flow region; and a second lane test line comprising immobilized test antibody; and measuring signal corresponding to the detection antibody at both the first lane test line and at the second lane test line to determine both decomplexed and complexed analyte levels in a sample.

In some aspects the invention provides a lateral flow test strip for detecting analyte levels in a sample comprising: a sample application region; a conjugate region comprising a detection antibody that selectively associates with the analyte; a flow region; and a test line comprising immobilized test antibody, wherein the test line is narrower than the width of the test strip, and its length to width ratio y to x is greater than 2:1, where the length y is parallel to the direction of flow. In some cases the length to width ratio y to x of the test strip is greater than 3:1. In some cases the length to width ratio y to x of the test strip is greater than 5:1. In some cases the length to width ratio y to x of the test strip is greater than 10:1.

In some aspects, the invention provides a lateral flow test strip for determining analyte levels in a sample comprising: a sample application region; a conjugate region comprising a detection antibody that selectively associates with the analyte; a flow region; and a test line comprising immobilized test antibody, wherein the test line comprises a plurality of test regions, each of the regions comprising the same test antibody. In some cases the test line comprises from about 4 to about 100 test regions. In some cases the test line comprises an array of test regions. In some cases the array of test regions is an array of n by p regions where n and p are independently 2, 3, 4, 5, 6, 7, 8, 9, or 10.

In some aspects the invention provides a lateral flow test strip for determining analyte levels in a sample comprising: a sample application region; a conjugate region comprising a detection antibody that selectively associates with the analyte; a flow region; and a test line comprising immobilized test antibody, wherein the test line comprises at least two portions, a high sensitivity portion having a length to width ratio y to x that is less than 2:1, and a high dynamic range portion having a length to width ratio y to x that is greater than 2:1, wherein the length y is in the direction of flow. In some cases the high sensitivity portion has a length to width ratio y to x that is less than 3:1, and a high dynamic range portion having a length to width ratio y to x that is greater than 3:1. In some cases the high sensitivity portion has a length to width ratio y to x that is less than 5:1, and a high dynamic range portion having a length to width ratio y to x that is greater than 5:1. In some cases the detection antibody comprises a fluorescent label.

In some aspects the invention provides a lateral flow assay method for detecting analyte levels in a sample with a reduced Prozone effect comprising: providing a lateral flow assay test strip comprising: a sample application region; a flow region; and a test line comprising immobilized test antibody; adding a solution comprising the sample to the strip, whereby the sample flows up the strip toward the test line; subsequently adding a solution comprising a detection antibody that selectively associates with the analyte; whereby the sample reaches the test line prior to the arrival of the detection antibody.

In some aspects the invention provides a lateral flow assay test strip for detecting analyte levels in a sample providing for a reduced Prozone effect comprising: an elution reagent addition region;

a portion of the strip downstream of the elution reagent addition region having a sample lane and a conjugate lane, wherein the sample lane comprises a sample application region, and

the conjugate lane comprises a conjugate region, comprising a detection antibody that selectively associates with the analyte; a flow region, and a test line comprising immobilized test antibody, the test strip configured such that sample is added to the sample addition region, and elution reagent is added to the elution reagent addition region, whereby the elution reagent flows down both the sample lane and the conjugate lane, and the rate of travel down the strip for the detection antibody in the conjugate lane is slower than the rate of travel down the strip for the sample in the sample lane, whereby the sample reaches the test strip before the detection antibody reaches the test strip. In some cases the conjugate lane comprises an altered fluid flow path. In some cases the conjugate lane comprises a serpentine flow path. In some cases the altered fluid flow path is produced by interdigitated hydrophobic barrier lines.

In some aspects the invention provides a lateral flow test strip for detecting analyte levels in a sample having improved sensitivity comprising: a sample application region; a decomplexation region comprising a decomplexation reagent for dissociating analyte-antibody complexes in the sample; a conjugate region comprising a detection antibody that selectively associates with the analyte; a flow region; and a test line comprising immobilized test antibody, wherein the width of the flow path in the lateral flow test strip at the test line is 80% or less of the width of the flow path at the sample addition region. In some cases the width of the flow path at the test line is 50% of the width of the flow path in the sample addition region. In some cases the width of the flow path at the test line is 20% of the width of the flow path in the sample addition region.

In some aspects the invention provides a portable fluorescent reader for detecting analyte levels in a sample, comprising: an illumination source providing excitation light; illumination optics for directing the illumination light to a lateral flow test strip; a region for holding a lateral flow test strip of any of the above claims, the lateral flow assay strip comprising a test line; light collection optics for directing light emitted from the test line on the lateral flow test strip to a detector; and a detector comprising a camera. In some cases the detector comprises a cell phone. In some cases the portable fluorescent reader further comprises a processor for analyzing data from the camera. In some cases the illumination light comprises an LED.

### BRIEF DESCRIPTION OF THE DRAWINGS

FIG. 1 shows a typical sandwich immunoassay and use thereof.

FIG. 2 shows a test strip comprising a decomplexation region and a neutralization region.

FIG. 3 illustrates expected results from some test strips with free and complexed analyte.

FIG. 4 A-G show different configurations of test strips with different mechanisms to implement decomplexation regions.

FIG. 5 shows a test strip with an exothermal heat disassociation mechanism.

FIG. 6 A-D show different configurations of test strips with different arrangements of decomplexation regions.

FIG. 7 shows a test strip with an exothermal heat disassociation mechanism.

FIG. 8 A-B show a test strips with an external heater disassociation mechanisms.

FIG. 9 A-B show different configurations for dual test strips for testing complexation levels.

FIG. 10 A-C show a typical sandwich immunoassay and use thereof.

FIG. 11 A-G show various test strip arrangements which allow for improved dynamic range, sensitivity, or combinations thereof.

FIG. 12 A-D show different test strips with and without flow shaping mechanisms.

FIG. 13 shows a test strip with a mechanism to minimize the prozone effect.

FIG. 14 shows an off-axis illumination system.

FIG. 15 shows results from test strips of utilizing free and complexed analyte.

FIG. 16 shows results from tests of signal and nonspecific binding of dyes.

FIG. 17 shows a table of signal to nonspecific binding ratios for various dyes.

FIG. 18 shows a portable lateral flow assay reader.

FIG. 19 shows images and plots resulting from a fluorescence lateral flow assay.

FIG. 20 shows images and plots resulting from an absorbance lateral flow assay.

FIG. 21 shows images and plots resulting from a fluorescence lateral flow assay dilution series.

FIG. 22 shows images and plots resulting from an absorbance lateral flow assay dilution series.

FIG. 23 A-B graphically show photobleaching studies for various dyes.

### DETAILED DESCRIPTION OF THE INVENTION

In some aspects, the instant invention provides test strips, systems, and methods for performing lateral flow assays. In particular, the invention relates to measuring the presence and/or level of analytes that are complexed in the sample that is added to the test strip, and therefore not accurately measured using conventional lateral flow assays.

Detection by conventional lateral flow methods of some clinically relevant targets can be hindered by association of these targets by complexing agents in the sample, such as antibodies that form analyte-antibody complexes. In conventional lateral flow assays, these complexes effectively shield the target analyte from reaction with interrogating test components, inhibiting detection of the analytes. Previous workers have shown that solution phase dissociation of these complexes can result in improved detection and quantitation of analytes. While pre-treatments such as these have been shown to provide better quality analyses, it would be desirable not to have to perform these extra steps.

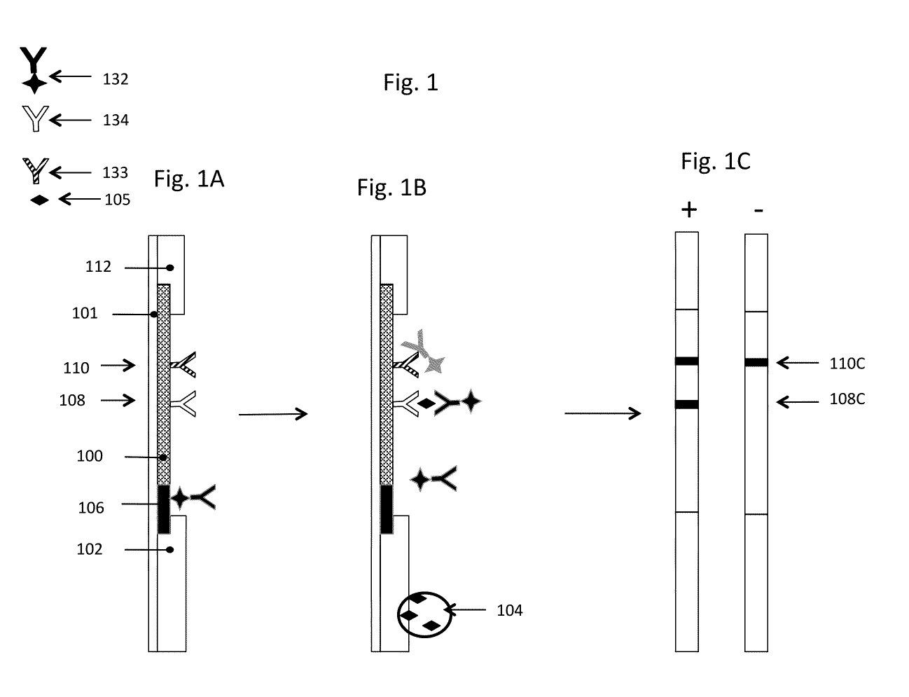

We have found that decomplexation of an analyte of interest can be accomplished on a lateral flow test strip, allowing for high quality analysis on a lateral flow strip without an extra, hands-on pre-treatment step. We describe herein how a lateral flow strip (a test strip) and reaction components can be modified to enable dissociation of antibody/analyte (antibody/antigen) immune complexes on the strip itself, providing access, binding, and target detection. FIG. 1 shows a typical sandwich immunoassay. The system describes uses human chorionic gonadotropin (hCG) test strips along with goat polyclonal anti-hCG. Lateral flow assays can be used for a wide range of antibodies and analytes. The strip is composed of sticky backing 101 to which is attached layers of membrane or substrate 100 which may be nitrocellulose, wicking pad 112, conjugate region wherein conjugate material may be applied to a conjugate pad 106 material and or a sample pad (glass fiber) material. Gold-labeled mouse anti-hCG 132 is dried on the conjugate pad 106; unlabeled mouse anti-HCG 134 is applied to the test region 108, and goat anti-mouse MAb 133 is applied to the control region 110 on the nitrocellulose membrane or substrate 100. To run the test, the fluid sample 104 is applied to the sample pad 102. Eluant may be applied with the fluid sample 104 or as a separate solution. Flow is upward in the diagram. The gold-labeled mouse anti-hCG 132 is released from the conjugate pad 106, forming a sandwich of surface bound polyclonal unlabeled anti-hCG 134, target analyte 105 hCG, and the labeled mouse anti-hCG 132 at the test line 108C if the target analyte 105 hCG is present. The presence of a band at the control region 110C indicates the assay is working properly.

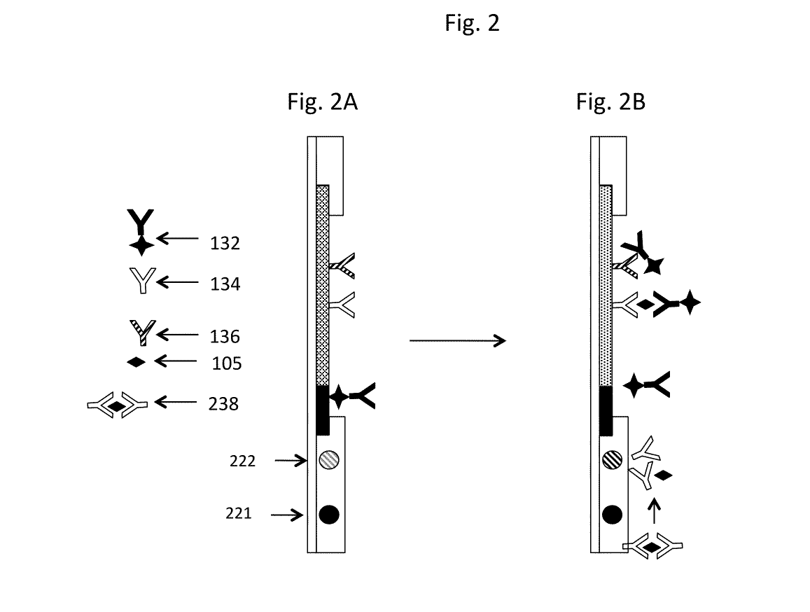

FIG. 2, which uses the same symbolic references as used in FIG. 1 illustrates how a decomplexation region can be used to denature immune complexes and provide for better measurements of analyte levels. Some or all of the target analyte 105 in the sample is complexed in an unlabeled immune complex 238, for example with antibodies in the patient. As the unlabeled immune complex 238 travels up the strip, the decomplexation region 221, for example using decomplexation reagents, denatures the complex, dissociating the analyte from antibodies present in the sample that are blocking the analyte from detection at the test region of the strip. As the sample continues up the strip, the neutralization region 222 neutralizes the decomplexation reagents, preventing them from interfering with downstream interactions on the strip. For example, neutralization reagents in the neutralization region are released, allowing binding of the analyte to the test region where it is detected. In some embodiments of the invention, the neutralization region is omitted, for example in case where a bound antibody effectively binds an analyte in the presence of the decomplexation reagents necessary to decomplex the native unlabeled immune complex 238.

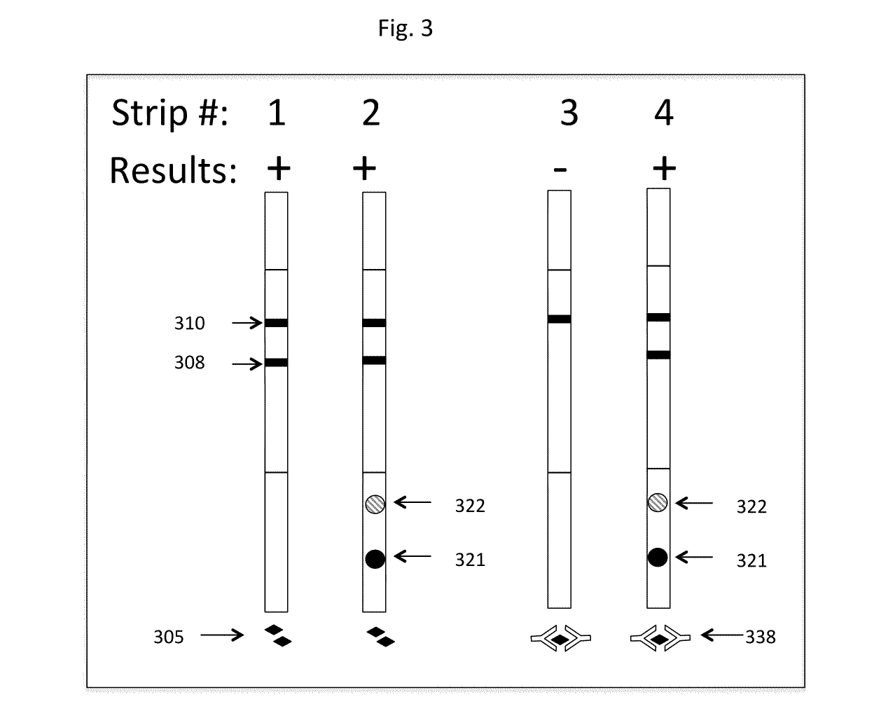

FIG. 3, which uses the same symbolic references as used in FIG. 1 shows results expected from test strips tested with free and complexed analyte, and with and without a decomplexation region 321 and neutralization region 322 on the strip. The test region 308 detects the presence of the analyte and the control region 310 acts as a control: Strip 1: free target analyte 105, without decomplexation region 321 and neutralization region 321; strip 2: free target analyte 105, with decomplexation region 321 and neutralization region 322. Since the analyte is not complexed the strip 1 and 2 provide the same answer; strip 3: complexed unlabeled immune complex 338 analyte, without decomplexation region 321 or neutralization region 322; strip 4: complexed unlabeled immune complex analyte, with decomplexation region 321 and neutralization region 322. Here, due to the decomplexation of the complexed unlabeled complex 338 analyte, a more accurate measure of the actual analyte in the sample is obtained. In addition, one can run the same sample with and without decomplexation as in strips 3 and 4 to provide a measure of how much complexation is occurring, which can be useful, for example, in understanding the progress of a disease. While the description above relates to hCG, it is understood that the methodology here can be used for a wide variety of types of antigens. A decomplexation reagent may in some cases be an acidifying or acidification reagent.

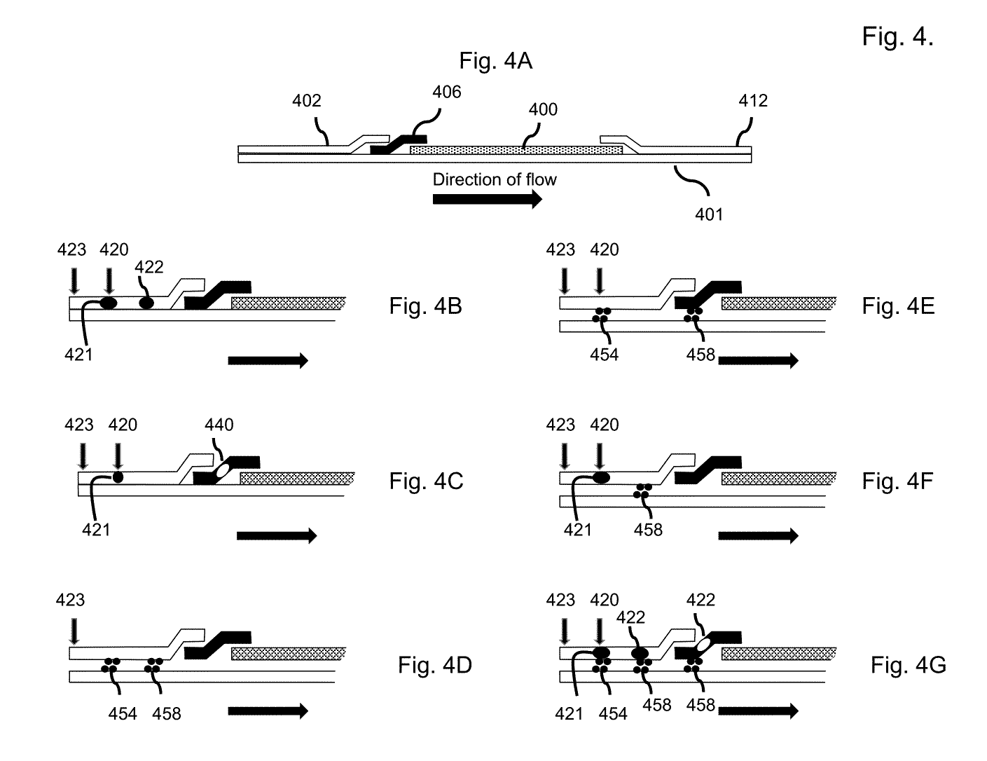

FIG. 4 shows various approaches to implementing the decomplexation region in a lateral flow test strip. It is understood that these are only some of the possible approaches, and that combinations of the approaches described are anticipated as part of the invention. FIG. 4A shows the components of a typical test strip. The strip has, a backing 401, a sample pad 402 onto which the sample and/or the eluent or elution reagent is added, a conjugate pad 406 which typically has labeled detection antibody applied thereto, a membrane or substrate 400 which may be a nitrocellulose film, down which the sample and eluent travel, the membrane or substrate 400 which may be nitrocellulose typically having a capture (test) line (not shown) and a control line (not shown). At the end of the strip is an absorbent wicking pad 412 to promote the wicking of the sample and eluent. FIGS. 4B through 4G show the beginning, or upstream, portion of the test strip.

FIG. 4B illustrates a strip configured to provide decomplexation and neutralization using soluble reagents that are deposited onto the strip. At decomplexation region 421 is dried down a soluble acid compound such as citric acid. At neutralization region 422 is dried down a soluble neutralizing agent, for example a soluble base such as Tris. The sample, at a volume of for example 5 to 20 microliters is added at sample input or sample addition region 420, after which elution reagent, e.g. elution buffer is added to eluent or elution reagent input region 423, for example at a volume of form 30 to 100 microliters. The sample in this embodiment is added directly onto the acid, allowing for the acid driven decomplexation of the complexed analyte in the sample. The elution reagent subsequently washes the sample past the neutralizing agent 440, which may be basic. Both the acidifying agent and the neutralization agent 440 are soluble in solution, and react to such that the decomplexed analyte solution is at the appropriate pH, e.g. around neutral pH when as it travels down the rest of the strip.

FIG. 4C illustrates another approach to using soluble decomplexation and neutralizing agents. In this approach, the soluble neutralization agent 440 is in the conjugate pad 406. In this way, neutralization occurs simultaneously with the exposure of the sample to the detection antibody. This approach allows for a longer time for the sample to be in contact with the decomplexation agents while it is in the strip.

FIG. 4D illustrates an approach to decomplexation utilizing insoluble decomplexation and neutralization reagents. In this embodiment, an insoluble decomplexation agent 454 such as a cation exchange resin in its acidic or protonated form is between the base and the sample pad. For example, the cation exchange resin, which is a solid material, is deposited onto the backing and sandwiched between the backing 401 and the sample pad 402, which can be made, for example, of glass fiber. Another approach is to embed the exchange resin in powder form into the glass fiber of the sample pad 402. Further down the strip is an insoluble neutralizing agent 458, for example anion exchange resin in its basic form. In this example, sample is added dissolved in the eluent at eluent input region 423 at a volume of e.g. 30 to 100 microliters. Alternatively, one could utilize a dipstick approach by immersing the end of the strip at about eluent input region 423 into a larger volume of solution, e.g. 0.1 mL to 50 mL. An eluent input region may be referred to as an elution reagent application region or an eluent application region.

FIG. 4E illustrates another approach to decomplexation and neutralization with solid agents. Here, the insoluble neutralization reagent is located at the conjugate pad as recited above for the soluble reagents. Here, the sample is added onto the insoluble decomplexation agent 454 at sample addition region 420. The elution buffer is added to elution input region 423, for example at a volume of form 30 to 100 microliters. The sample in this embodiment is added directly onto the acid, allowing for the acid driven decomplexation of the complexed analyte in the sample. The elution reagent subsequently washes the sample past the insoluble neutralizing agent 458. A dipstick method could also be used. A sample addition region may be referred to as a sample input region or a sample application region.

FIG. 4F illustrates an approach in which a soluble decomplexation agent is applied to a decomplexation region 421 and an insoluble neutralizing agent 458 are used. Here, the soluble neutralizing agent can be a detergent, salt such as sodium chloride, or a chaotropic agent such as urea. The insoluble neutralizing agent 458 could be a gel filtration medium.

FIG. 4G illustrates how a combination of decomplexation agents and neutralization agents can be used. As described herein, in some cases, combinations of decomplexation agents can be more effective than a single agent at effecting decomplexation. In this embodiment, both a soluble decomplexation reagent is applied to a decomplexation region 421, such as an acid, detergent, chaotropic agent or salt) is used along with an insoluble decomplexation agent 454 such as ion exchange resin. The strip has a first neutralization region 422 with both a soluble neutralizing agent and an insoluble 458 neutralizing agent. The strip also has a second neutralization region 422, also with both a soluble and an insoluble neutralizing agent 458. This approach can provide for a strong decomplexation, followed by a thorough two step neutralization. While described for a type of decomplexation and neutralization agent, the examples above can be applied to any suitable decomplexation or neutralization reagent such as those described herein.

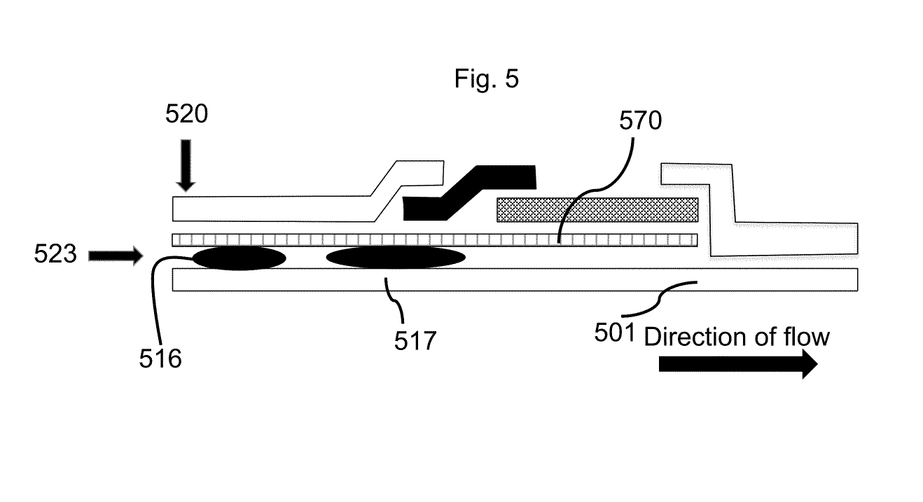

FIG. 5 illustrates a test strip that provides heat decomplexation in which the heat is provided by the interaction of the sample and/or eluent fluids with exothermic compounds in the strip. The test strip has exothermic reagents 516 which can be salts such as calcium oxide on top of the backing 501. This creates a decomplexation region near the beginning or upstream portion of the strip. The sample may also have region with endothermic reagents 517, which may comprise salts, further up the strip if required to cool the sample before it reaches the conjugation pad. In one approach, buffer is first added, for example, at eluent or elution reagent input region 523, which begins to heat the exothermic salts. The sample is then added at sample input or sample addition region 520 and eluted over the heated region. In some cases, a one step addition of a mixture of sample and elution reagent can be made at 520 or 523. A liquid impermeable membrane 570 which has good heat transfer characteristics can be employed to allow for transfer of heat without exposing the sample to the exothermic salts. The membrane 570 can also be a membrane that allows the passage of water into the salts below, but does not allow passage of the larger components of the sample and eluent solutions, such as antibodies or target proteins or nucleic acids.

Many exothermic salts are known. Suitable exothermic salts that provide heat when coming into contact with aqueous solutions include calcium oxide, copper sulfate, calcium chloride, and sodium carbonate. Suitable endothermic salts for cooling the eluent on the strip include potassium chloride, ammonium nitrate, sodium thiosulfate, ammonium chloride, urea, and sodium bicarbonate.

### Decomplexation Region

The decomplexation region on the strip is designed to provide the reagents or conditions for decomplexation or dissociation of the analyte-antibody complex. It has been shown by others that a pre-treatment of the sample can provide the level of decomplexation necessary to free the analyte for a more accurate determination of analyte levels in the sample. One aspect of the invention is the incorporation of these decomplexation reagents and methods onto the test strip itself by providing a decomplexation region, along with optional neutralization region, that alters the chemical or physical characteristics of the sample in order to provide decomplexation and free the analyte. These decomplexation methods have been used to decomplex antigens in solution, prior to analysis, for example with an ELISA test. Such decomplexation methods are described, for example, in U.S. Pat. No. 8,263,415 Sep. 11, 2012, U.S. Pat. No. 6,706,486 Mar. 16, 2004, U.S. Pat. No. 5,689,393 Dec. 16, 1997, U.S. Pat. No. 5,654,156 Aug. 5, 1997, U.S. Pat. No. 5,571,723 Nov. 5, 1996, U.S. Pat. No. 5,556,745 Sep. 17, 1996, U.S. Pat. No. 5,484,706 Jan. 16, 1996, U.S. Pat. No. 5,073,485 Dec. 17, 1991, U.S. Pat. No. 5,061,790 Oct. 29, 1991, U.S. Pat. No. 4,950,612 Aug. 21, 1990, U.S. Pat. No. 4,900,684 Feb. 13, 1990, U.S. Pat. No. 4,752,571 Jun. 21, 1988, U.S. Pat. No. 4,703,001 Oct. 27, 1987, U.S. Pat. No. 4,658,022 Apr. 14, 1987, U.S. Pat. No. 4,459,359 Jun. 10, 1984, U.S. Pat. No. 4,299,815 Nov. 10, 1981, and p24 Analyte Rapid Test for Diagnosis of Acute Pediatric HIV infection Z. A. Parpia et. al. Journal of Acquired Immune Deficiency Syndrome Volume 55, Number 4, Dec. 1, 2010 which are incorporated by reference herein in their entirety for all purposes.

For example, it has been found that the accurate measurement of p24 for understanding and treating HIV is compromised by decomplexation, and that assays for p24 (e.g. in an ELISA test) can be significantly improved by a prior decomplexation step. See, for example, International Patent Application WO2014039561 which is incorporated herein by reference for all purposes. Not all p24 proteins in a sample are extraviral and p24 proteins that are associated with intact viruses are usually not detectable. Moreover, in seroconverted individuals, extraviral p24 is predominantly immunocomplexed and generally unavailable for capture in p24 immunoassays. To improve the sensitivity of p24 assays, samples may be subject to treatment by detergents and heat, or by acid followed by neutralization, to release p24 from both viral particles and anti-p24 antibodies. See e.g., Schupbach et al. (2006); Nishanian et al. (1990); and Schupbach et al. (1996). For example, the commercial p24 ELISA kit from Perkin Elmer® uses a detergent and neutralization approach for immune complex disruption. Parpia et al. (2010) describe a method in which heat shock is used to improve p24 antigen detection sensitivity in a rapid test format. Methods that use chemical or heat decomplexation, however, can lead to denaturation of sample antibodies, compromising the ability to detect both antigen and antibody in a sample. For example, decomplexation methods applied to blood, serum, or plasma from HIV-infected individuals may compromise the antibody detection aspect of the fourth-generation assay, or associated antibody detection based co-infection serology assays. In an embodiment, the present disclosure provides a method for disrupting the viruses which helps increase the detectable concentration of p24 without significantly compromising the ability of a fourth generation assay to also detect anti-HIV antibodies. In some cases, the decomplexation region delivers reagents into the sample solution that promote decomplexation.

Any suitable reagent can be used. For example, reagents can change the acidity of the sample, raise the salt level in the sample, provide detergents, chaotropic agents, or organic solvents or a combination of any of these. In some cases, the decomplexation region changes the physical characteristics of the sample to promote decomplexation. For example, the temperature within a region of the test strip can be raised, which is known to promote decomplexation. The decomplexation reagent can be solid, or liquid. The decomplexation reagent can be a polymeric reagent. In some cases, the decomplexation reagent can release components into the sample and/or elution reagent to promote decomplexation. In some cases, the decomplexation reagent can be water soluble, in other cases, the decomplexation reagent can remain primarily on the test strip. In some embodiments, a combination of regents and changes in physical characteristics such as temperature may be utilized in a decomplexation region.

As recited above, in some assays, access to target moietie(s) can be inhibited as a result of inaccessibility of the target to bound binding moieties or labels which might otherwise bind to the target moietie(s) as a result of complexation of the target with other moieties in the raw sample. For example a target moiety can be an antigen which may be complexed with an antibody in the raw sample. The antibody may bind in a location wherein the antibody may block or inhibit the binding of a label or a bound binding moiety to the target moietie(s).

Thus as described herein, it can be desirable to disrupt the complex, which may be a complex between an antibody and a target moiety, or may be a complex between a target moiety and any other moiety which may render the target less accessible to a label or a bound binding moiety. Disruption may be effectuated utilizing changes in buffer conditions, which may include changes in pH, and may be combined with changes in temperature, such as increases in temperature. In some embodiments chemicals may be utilized to disrupt analyte complexes such as antibody complexes that reduce the active analyte concentration.

In some embodiments, the complex can be disrupted prior to adding a sample, which may include target moieties, to a strip, or as part of a lateral flow assay. In some embodiments, complexes may be disrupted by modification of pH, particularly by changing the pH to an acid pH, such as a pH between 3.5 and 3.0, a pH between 3.0 and 2.5, a pH between 2.5 and 2.0, or a pH less than 2.0. Changing a pH from a higher pH to a lower pH may be referred to as lowering a pH. Changing a pH from a lower pH to a higher pH may be referred to as raising a pH.

After disruption of complexes, it can be desirable to again change the conditions, which can include buffer conditions which can include pH and or temperature to conditions which can better permit binding of bound binding moieties or labels to target moieties. Thus it can be desirable to add a base or buffer, and to reduce the temperature so as to create conditions which can be suitable for binding of any labels or bound binding moieties. We typically refer to these conditions as neutralization conditions. A reason for neutralization of the decomplexation conditions is that it can be undesirable to have the decomplexation conditions present when the analyte is passing through the conjugate pad, as the decomplexation conditions can, in some cases, lower or prevent the binding of the detection antibody to the analyte.

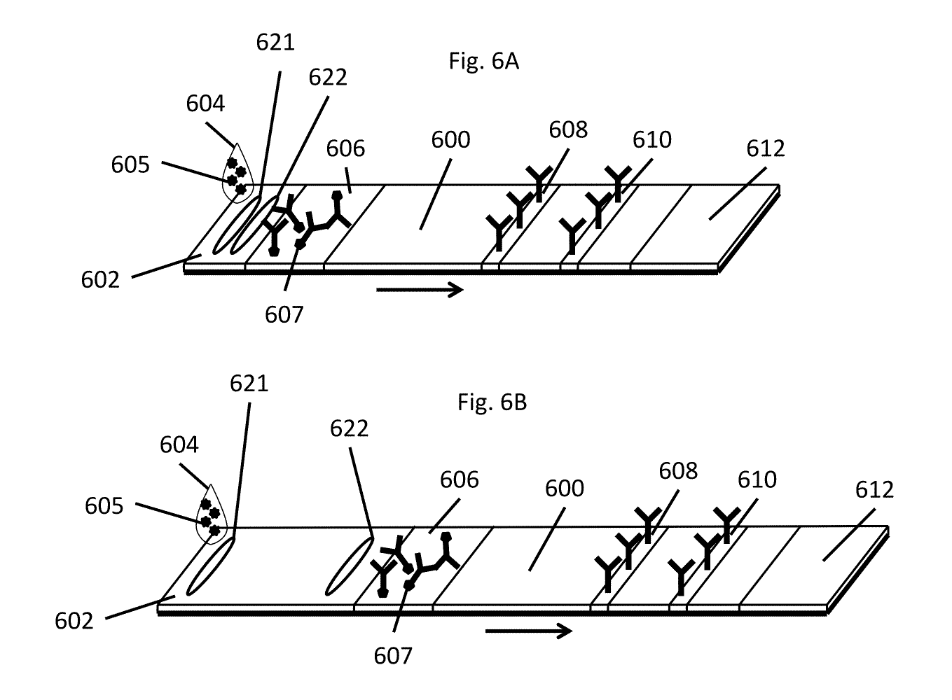

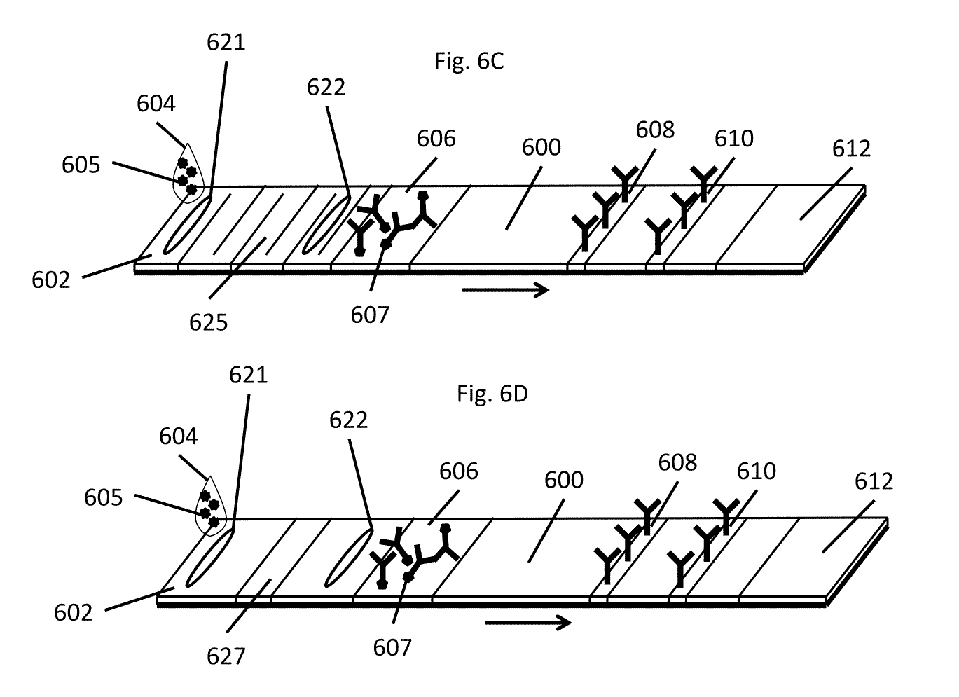

In some embodiments, pH can be modified at least in part by adding a buffer to target sample prior to applying a target sample to a lateral flow assay. In other embodiments as illustrated in FIG. 6A, a fluid sample 604, which can be a clinical sample fluid containing target analyte 605, potentially a complexed or partially complexed analyte, can be applied to a sample pad 602 which can be partly overlapping conjugate pad 606 which overlaps the substrate or membrane 600, which can be a nitrocellulose membrane. A conjugate pad 606 can have labeled antibodies specific to the target analyte 607, wherein a pH change can be effectuated by applying an acid to an decomplexation region 621 of a sample pad 602; acid or other decomplexing reagents can be applied to the decomplexation region 621 and can be dried as part of a manufacturing process; similarly as illustrated in FIG. 6A, a pH change can be effectuated by applying a base and or buffer to an neutralization region 622 of a sample pad 602; the base and or buffer can be applied to the neutralization region 622 and can be dried as part of a manufacturing process. A test region 608 comprising antibodies specific to the target analyte as described herein can be bound to the substrate or membrane 600 positioned after the conjugate pad 606 so that decomplexed analyte can interact with the labeled antibodies specific to the target analyte 607 prior to interacting with the antibodies bound at the test region 608. A control region 610 comprising antibodies specific to the Fc region of the labeling antibody as described hereinabove can be bound to the substrate or membrane 600 positioned such that the sample will interact with the test region 608 prior to interacting with the control region 610. A wicking pad 612 can be provided, which can be adjacent to or overlapping part of the substrate or membrane 600, and can provide a volume to enable substantially all of the fluid sample 604 which may be a clinical sample fluid containing now decomplexed target analyte 605 to pass by and interact with the test region 608.

In some embodiments, an acid applied to an acid or decomplexation region can be a nonvolatile water soluble compound which can comprise a carboxylic acid group and or a sulfonic acid group, wherein the acid can have an R_{f} on the membrane of from 0 to 1.0. In some embodiments, a weak acid can exchange a hydronium ion for an ion already in solution, such as for example, a sodium ion, thereby not increasing the ionic strength of the solution, and further not retaining or binding proteins which can be in the fluid. Examples of weak acids with an R_{f} of close to 1 can include citric acid, oxalic acid, and ascorbic acid.

When a sample is added to a sample pad, the sample can thence flow towards the decomplexation region 621 dissolving the acid and changing the pH of the fluid in the vicinity of the decomplexation region 621. The acidified sample fluid can continue to flow, and can interact with base and or buffer in a neutralization region 622, whereby the pH of the target fluid can be increased to a pH suitable for binding of a label or bound binding moiety.

In some embodiments, it can be desirable to allow a period of time to pass so as to permit disruption of any complexes to be more fully effectuated. This can require a longer period of time than can be permitted with a closely spaced decomplexation region 621 and neutralization region 622. Thus in some embodiments as illustrated in FIG. 6B, it can be desirable to utilize a longer sample pad 602 than might otherwise be utilized. The spacing between an decomplexation region 621 and a neutralization region can be between less than two millimeters, two and five millimeters, between five and ten millimeters, between ten and twenty millimeters, between twenty and forty millimeters, or more than forty millimeters.

In some embodiments, the time utilized for disruption of complexes can be increased by increasing the hydrophobicity of the sample pad 602; the acidified target fluid can thus flow more slowly between a decomplexation region 621 and a neutralization region 622.

In some embodiments, more time can be needed for disruption between a decomplexation region 621 and a neutralization region 622 than can be reasonably permitted by a reasonably sized sample pad 602; the amount of fluid sample 604 needed can also be excessive. Thus in some embodiments as shown in FIG. 6C, it can be desirable to utilize a serpentine region 625 or other similar shape between an complexation region 621 and a neutralization region 622, wherein the cross section of the serpentine region 625 can be smaller than the cross section of the sample pad 602 and or other regions of the membrane or substrate 600, and the fluid path length between the decomplexation region 621 and the neutralization region 622 can be extended without requiring additional fluid sample 604.

In some embodiments as shown in FIG. 6D, an decomplexation region 621 can be separated from other portions of a lateral flow assay by a meltable wax region 627, wherein wax in the meltable wax region 627 cannot melt at temperatures below a particular temperature, which can be a temperature below a temperature sufficient to denature proteins which can comprise target analyte 605. Decomplexation region 621 can be heated to a temperature below that needed to melt wax in meltable wax region 627 for a period of time as needed for decomplexation of target analyte 605, wherein said period of time can be from one to five minutes, from three to fifteen minutes, from ten minutes to an hour. The temperature of the meltable wax region 627 can thence be raised to a temperature sufficient to melt wax in said meltable wax region 627, permitting now decomplexed target analyte to pass down the lateral flow assay and subsequently interact with a test region 608. A meltable wax can be chosen so as to not interfere with the interactions of a target analyte 605, labels specific to the target analyte 607, or between target analyte 605 and test region 608. In other embodiments, multiple antibodies can be utilized to bind to antigens. As wild type antibodies which can be complexed a desired target antigen can be polyclonal, it can be desirable to utilize multiple antibodies to bind antigens which have complexed with native antibodies, such that a target antigen can be bound in a number of locations on the surface of the target antigen. In some embodiments, multiple antibodies can be bound within a single binding region; in other embodiments, multiple antibodies can be bound to different individual labels, wherein the different labels can be the same species of label, or can be different types of labels; in further embodiments, multiple antibodies can be bound to a single label species, wherein the different labels can be bound utilizing linkers.

### Decomplexation Agents

A decomplexation agent is generally an agent which, when present in the decomplexation region, or is released from the decomplexation region, results in the release of the analyte or antigen from the complexing agents which are binding to it and preventing its detection. There are many types of decomplexation agents. Typically the complexation agents are proteins such as antibodies, and agents that can disrupt a protein interaction with an analyte can be used as decomplexation agents. In some cases the analyte is also a protein, and therefore agents that disrupt protein-protein interactions can act as decomplexation agents. Decomplexation agents include acids, alkylating agents, salts, detergents, chaotropic agents, and organic solvents. It is understood that these categories are not mutually exclusive, and therefore a chaotropic agent may be an organic solvent, and a detergent may be an acid or a salt. These categories are provided as a guide for selecting the appropriate decomplexation agent for the application. The selection of the appropriate decomplexation agent can be done with standard experimental approaches.

### Acid

In some cases, the decomplexation region acidifies the sample or sample and elution reagent or elution buffer in order to promote decomplexation. Suitable acids include, for example, citric acid, glycine-HCl, benzene sulfonic acid, succinic acid, maleic acid, and tartaric acid. In some cases the acids are polymeric acids, such as polymeric cation exchange materials in their protonated or acid form. Acids comprising carboxylic, sulfonic, phosphonic, and phosphate groups can be used. Chaotropic agents, including acids that act as chaotropic agents can also be used. Suitable chaotropic agents include trifluoroacetic acid and peroxy acids. In some cases, the pH of the sample or sample/elution buffer is brought to below pH 5. In some cases, the pH of the sample or sample/elution buffer is brought to below pH 4. In some cases, the pH of the sample or sample/elution buffer is brought to below pH 3. In some cases, the pH of the sample or sample/elution buffer is brought to below pH 2. In some cases a rise in temperature is combined with a lowering of pH to promote decomplexation.

### Alkylating Agents

In some cases alkylating agents can be used. The alkylating agent can react, for example with the complexing agents such as antibodies in the sample in order to promote decomplexation. In some cases, alkylating agents are chosen to react with the complexing agents in the sample while reacting minimally with the analyte so the analyte is still detectable on the strip. Suitable alkylating agent include gluteraldehyde, O-methylisourea, formaldehyde, butanedione, cyclohexanedione, or other agents which result in decomplexation by modifying lysine, argentine, or primary amine groups of interfering antibodies.

### Salts

In some cases, the decomplexation region provides salt into the sample or sample and elution reagent or elution buffer in order to promote decomplexation. In some cases, the appropriate salt can be dried down into the decomplexation region for release by solubilization into the sample. Suitable salts include magnesium chloride, lithium chloride, and sodium thiocyanate.

### Detergents

The decomplexation region can provide detergents into the sample or sample and elution reagent or elution buffer in order to promote decomplexation. Suitable detergents include nonionic detergents such as Nonidet P40, Tween 20, and Triton X-100, zwitterionic detergents such as CHAPS, and CHAPSO, anionic detergents such as sodium dodecyl sulfate (SDS), and cationic detergents such as benzalkonium chloride and alkyl trimethylammonium bromide.

### Chaotropic Agents

The decomplexation region can provide chaotropic agents into the sample or sample to promote decomplexation. Chaotropic agents are typically molecules in water solution that can disrupt the hydrogen bonding network between water molecules. This has an effect in the stability of the native state of other molecules in the solution, mainly macromolecules (proteins, nucleic acids) by weakening the hydrophobic effect. For example, a chaotropic agent reduces the amount of order in the structure of a protein formed by water molecules, both in the bulk and the hydration shells around hydrophobic amino acids, and may cause the denaturation of proteins with these amino acids. Suitable chaotropic agents include guanidine-HCl, urea, lithium perchlorate, lithium acetate, magnesium chloride, phenol, butanol, ethanol, propanol, sodium dodecyl sulfate, and thiourea.

### Organic Solvents

The decomplexation region can provide organic solvents into the sample or sample and elution reagent or elution buffer to promote decomplexation. The organic solvent should typically be soluble in water, and have a low enough volatility to be stored on the test strip. Suitable organic solvents include ethylene glycol. In some cases an organic solvent can be molecularly encapsulated in a water soluble capsule. This allows for the organic solvent to be immobilized on the strip, but to be released, or provided into the aqueous elution reagent in order to promote dissociation. See, for example, Westdeutsche Zeitung, 28 Oct. 2004; and Le, et al., PDA journal of pharmaceutical science and technology/PDA 60 (5): 314-322 (2006) which are incorporated herein by reference.

### Heating

Heating is known to disrupt the analyte-antibody complexes. The invention includes test strips and systems where a portion of the strip is heated in order to promote decomplexation. Typically, the lateral flow assay is carried out at room temperature. In some embodiments of the invention, the bulk of the strip is kept at about room temperature, but the region of the strip below the conjugate region or conjugate pad is heated in order to promote dissociation of the antibody analyte complex. In some cases, the heated decomplexation region is coextensive with the sample addition region such that the sample is heated as it is added to the strip. In other cases, the heated region is between the sample addition region and the conjugate region. The sample and elution reagent then cools after passing through the heated region into the remainder of the test strip. The heated region may include one or more test lines, wherein binding agents such as antibodies are capable of binding under conditions including temperature which causes decomplexation of native complexes. Heating is also used in combination with other analyte-antibody disruptions approaches, such as those described herein. In some cases the heating of the sample is additionally used to facilitate a controlled temperature for better reproducibility than is obtained when relying on room temperature.

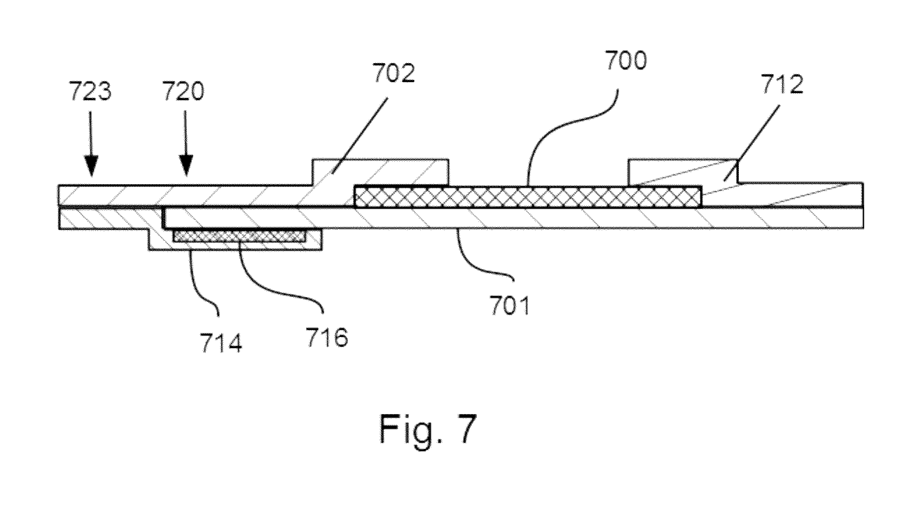

One aspect of the invention provides for providing heat in the decomplexation region by heat generated by the interaction of the sample and/or the eluent reagent and reagents on the decomplexation region. In this way, an exothermic reaction can provide heating for decomplexation without external heating sources. In some cases the exothermic chemical reaction can exist in the sample path either at the sample loading point or downstream of that point. In some cases as shown in FIG. 7, the exothermic chemical reaction can occur adjacent to but fluidically separated from some of, or the entire sample. In FIG. 7 the backing 701 can be any nonporous substrate such as plastic. Sample is first applied to the sample pad 702 at a point shown by the S in FIG. 7. When buffer is added to eluent input region 723 to the sample pad it also wicks through the exothermic reagent support pad 714. An optional conjugate pad (not shown) can be used upstream of the membrane or substrate 700. In some cases sample fluid may be used in place of buffer to activate an exothermic reagent. An exothermic reagent support pad 714 can be fabricated from a faster wicking material than the material typically used for a sample pad 702. Sample and or eluent may be drawn to a wicking pad 712. In alternative embodiments, a buffer may be added prior to adding a sample, such that said buffer reaches said exothermal reagent support pad 714 before sample has been applied to a sample input or sample addition region 720, or to a eluent input region 723, or to both a sample input region and a eluent input region 723, or before sample has passed said exothermal reagent support pad 714. When the buffer contacts the exothermic reagent 716, heat is generated, raising the temperature of the sample. Suitable exothermic reagents include calcium oxide, which can provide heat when brought into contact with an aqueous solution. A sample addition region may also be referred to as a sample input region or a sample application region.

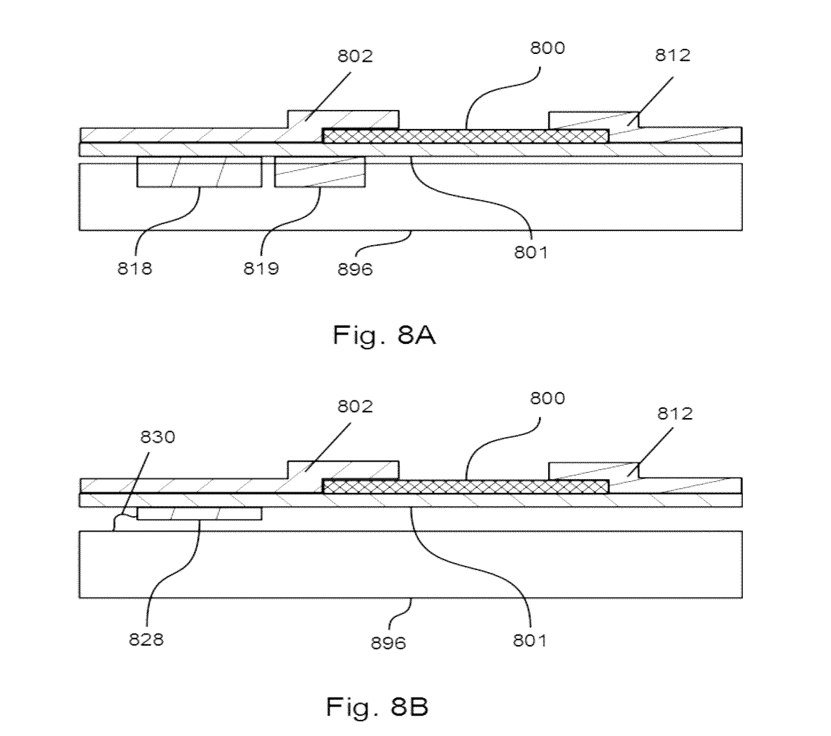

Other heating sources such as electrical heaters and infrared heaters can also be used. In some cases a heater is built into a lateral flow reader. As shown in FIG. 8A a heater 818 can be part of a lateral flow reader 896. Here the heater is held in thermal contact with the backing 801. The lateral flow device can comprise a sample pad 802, membrane or substrate 800 and wicking pad 812. Heater 818 can have discrete or surface mount resistors, resistive wire such as nichrome wire or kanthal wire, electrically conductive rubber, metal films, heaters, thermally conductive heat spreaders including metal plates, and the like. Backing 801 can be held in thermal contact with heater 818 by, for example, compression. In some cases a metal heat spreader can be used. In other cases, an optional heat sink 819 such as aluminum can be provided in the lateral flow reader 896 to allow cooling of the fluid after it passes a decomplexation region.

In some cases a temperature sensor can be used to provide feedback for temperature control. For example, current(s) and voltage(s) can be measured so that a controlled power level can be provided. In some cases the resistance of a heater element, which can have a known temperature coefficient can be measured and used to monitor or control the temperature; in other embodiments separate thermal sensors, which can be utilized to measure one or more of the ambient temperature and or one or more portions of a lateral flow device(s). Multiple heaters or heater regions can be utilized; in some embodiments multiple heating zones can be effectuated by utilizing one or more metal spreaders so as to couple a single heating element to multiple heating zones. For example, multiple heating regions each at different temperatures can be used, for example, one heating region is used to decomplex a sample target, while another heating region is utilized to maintain one or more test and or control lines at a set temperature, preventing variation in binding kinetics due to ambient temperature changes, and permitting a more reliable and quantitative binding. The heater regions, can also, in some cases provide cooling. In some cases the strip has a high temperature region for decomplexation, followed by lower temperature regions where the sample is cooled before the subsequent steps on the strip such as binding with the detection antibody.

The heating element, such as the resistive element can be part of the test strip. FIG. 8B shows a resistive element 828 that is part of a lateral flow strip. Suitable resistive elements include thin metallic or non-metallic films and electrically conductive paints or inks. In other cases resistive materials such as conductive rubber or plastic, which can be both thermally conductive and electrically conductive, are used. In other embodiments, a heater is utilized with a compliant material such as a compliant thermally conductive material so as to allow good thermal conductivity between parts which are not coplanar. Such resistive element(s) can be applied directly to a backing 801 or to a separate portion of the strip. Adhesive, which can be a thermally conductive adhesive, can be used to attach and provide good thermal contact between a heater and a backing support. A lateral flow reader 896 can be used to provide a current or voltage source for the resistive heating element via electrical connections 830.

In some cases light such as an infrared source can be used to provide local heating. The absorbance of the lateral flow device can be locally varied, such as with printed zones that absorb emitted radiation, to provide localize heating. The light can be focused or an aperture used to control the extent of the heating zone. Focusing can be obtained, for example, with a cylindrical lens to focus emitted energy into a line.

Typically the heating step is selected such that the analyte is not substantially denatured and its structure is effectively unchanged, but in some cases, the heating element can be used to improve the binding of the analyte. For example, in some cases, heating the analyte denatures it such that portions of the protein which had been inaccessible due to folding can now be utilized for subsequent binding either to surface bound capture antibodies or to label antibodies.

### Combinations

In many cases it is preferred to use combinations of the above methods for decomplexation. For example, a combination of heating and acidification, organic solvents and detergents, or high salt and acidification can be used.

### Neutralization Region

As described above, the decomplexation of the analyte-antibody complexes in the sample can be useful in releasing the analyte for detection. However, the same decomplexation reagents and conditions can also interfere with the subsequent analyte-antibody interactions on the strip that are required for detection. Thus, we have found that in addition to a decomplexation region, the strip in some cases is also provided with a neutralization region. This region neutralizes or soaks up the decomplexation reagent in order to prevent it from interfering, for example with the binding of the detection antibodies. In some cases, resins that can take up acid, detergents, salts, etc. can be used. For example, ion exchange resins can be employed. In the case of acid decomplexation agents, bases or buffers or ion exchange resins in their basic form can be deposited into the neutralization region to act to neutralize the acids. Suitable buffers include Tris buffer. For detergents as decomplexation agents, in some cases, Sephadex™ regions can be used for neutralization. Where salts are used for decomplexation, Sephadex™ or specific traps for the ions in the salts can be employed.

### Elution Reagent or Elution Buffer Provides Neutralization

In some aspects of the invention, the elution reagent or buffer can provide the neutralization of the decomplexation reagent that is required for removal of the decomplexation reagent from the downstream portions of the strip. For example, the elution buffer can have reagents that react with the decomplexation reagents for neutralization. One approach is to have a decomplexation region coextensive with the sample application region such that the sample is acidified resulting in decomplexation. The elution buffer, which may in this case be added subsequently, passes through these regions, bringing the sample up the strip for detection, while also neutralizing the acid used for decomplexation. Similar approaches can be used with the other decomplexation reagents recited herein. In some cases, neutralization can be accomplished or enhanced by dilution. In this way, the elution buffer can provide neutralization by diluting the decomplexation reagent to a level at which it will not interfere with the downstream analysis. In some cases, dilution can be enhanced by providing more than one channel for the passage of elution reagent or elution buffer, e.g. one or more parallel channels. In other embodiments, the sample may initially be added to a reagent mixture that includes acids, salts or other reagents which result in decomplexation. In some embodiments decomplexation may be effectuated by the use of a reagent mixture that comprises a salt which may raise the salt concentration of the environment of the analyte. Deleterious effects associated with the reagent mixture are then neutralized in the neutralization region of the strip.

### Dual Lateral Flow Detection

One aspect of the invention provides for measuring the level of analyte in a sample by measuring analyte levels with and without decomplexation. This can be done using two separate lateral flow devices, one providing decomplexation, and the other having no decomplexation. A preferred aspect of the invention provides for measuring analyte levels with and without decomplexation on the same test strip, referred to herein as a dual lateral flow device. The dual lateral flow device typically uses a common sample. The device can have a common buffer addition area to allow for a single addition of buffer for both decomplexed and non-decomplexed portions. As the sample travels up the strip, a portion of the sample is passed through a decomplexation region as described herein, and another portion of the sample does not experience decomplexation. In some cases, the two portions of the sample travel in physically separated lanes. The lanes can be fluidically separated by removing a portion of the membrane between the lanes. The lanes can be physically separated using fluid dams or barriers such as wax barriers, crush zones and the like. In some cases no physical barrier is used but instead the lateral flow of the sample allows for separate measurements to be made. For example, the measurements may be made sufficiently far apart that the linear flow of a lateral flow assay prevents significant diffusional mixing between the measurement regions. Where no fluidic barrier is used, a border zone between measurement regions can be ignored, or blocked by an imaging aperture (not shown).

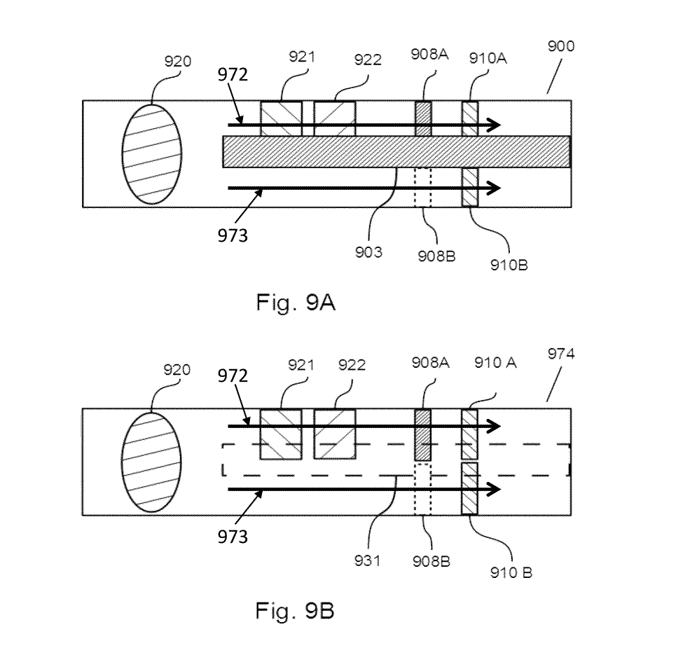

FIG. 9A shows a diagram of a dual lateral flow device with fluidic separation barrier 903 separating the added sample into two portions which proceed to analysis down separate lanes. In some embodiments a fluidic separation barrier may be effectuated by utilizing two separate membranes or substrates, while a single wicking pad, and a single sample input region may be utilized to form a single lateral flow test strip device. Sample input or sample addition region 920 is a region to which the sample and elution reagent (buffer) are added. Here both sample and eluent are added in the same region. In other cases, there can be separate regions for sample and elution reagent. Also, in some cases, the sample can be added with the elution reagent as described herein. The sample is eluted up the strip and proceeds down two lanes or separate flow path(s) 972 and 973 as indicated by the arrows. The portion of the sample in lanes or separate flow path 972 passes through decomplexation region 921 and neutralization region 922 and is detected at target region 908A. The strip also typically has control regions 910A and 910B to ensure that the strip is performing properly. The portion of the sample in lane or separate flow path 973 is eluted without experiencing decomplexation and is detected at target line 908B. Where there is strong complexation of the antigen in the sample, and decomplexation is effective, there will be a strong band at 908A representing the detection of the decomplexed antigen, and a weak band or no band at 908B because detection of analyte was prevented due to complexation. Being able to measure decomplexed and un-decomplexed analyte levels in this way is useful so as to measure the level of complexation of a sample. It is particularly useful to use this dual detection with quantitative detection, e.g. using a fluorescent lateral flow assay. The dual test strip also typically has a conjugate region in each of the lanes or separate flow paths (not shown). The conjugate region in lane 972 is located after the decomplexation region 921 and before the test strip 908A. If there is a neutralization region 922, the conjugate region can be located after the neutralization region, or in some cases, as described herein, the conjugate region can be coextensive with the neutralization region 922. The conjugate region in lane or separate flow path 973 can is located before test strip 908B. It is typically located directly across from the conjugate region in lane or separate flow path 972. The terms before and after refer to the position of the feature relative to the direction of flow.

FIG. 9B shows a diagram of a dual lateral flow device 974 without a physical separator. A border zone 931 may be optically blocked using an aperture, or may be ignored, either in image analysis, or by a user visually ignoring signal in the border zone 931.

The decomplexation region 921 and the neutralization region 922 can include any of the approaches described herein for accomplishing decomplexation and neutralization.

### Lateral Flow Assays

Any suitable lateral flow assays can be used with the invention. The invention can be used with sandwich assays and with competitive assays. A lateral flow assay is typically carried out on a lateral flow strip or test strip. Preferred lateral flow assays include those assays using fluorescent detection as describe in U.S. Provisional Patent Application 61/961,428, which is incorporated herein by reference in their entirety for all purposes. Lateral flow assays are described, for example in U.S. Pat. Nos. 5,770,460, 4,943,522; 4,861,711; 4,857,453; 4,855,240; 4,775,636; 4,703,017; 4,361,537; 4,235,601; 4,168,146; and 4,094,6478,003,407 Aug. 23, 2011, U.S. Pat. No. 5,753,517 May 19, 1998, U.S. Pat. No. 4,999,285 Mar. 12, 1991, and U.S. Pat. No. 4,361,537 Nov. 30, 1982, which are incorporated herein by reference in their entirety for all purposes. Lateral flow assays can be used to measure a variety of analytes from a large numbers of types of samples. The samples can include biological materials and fluid, and in humans can include, for example whole blood, serum, urine, or saliva.

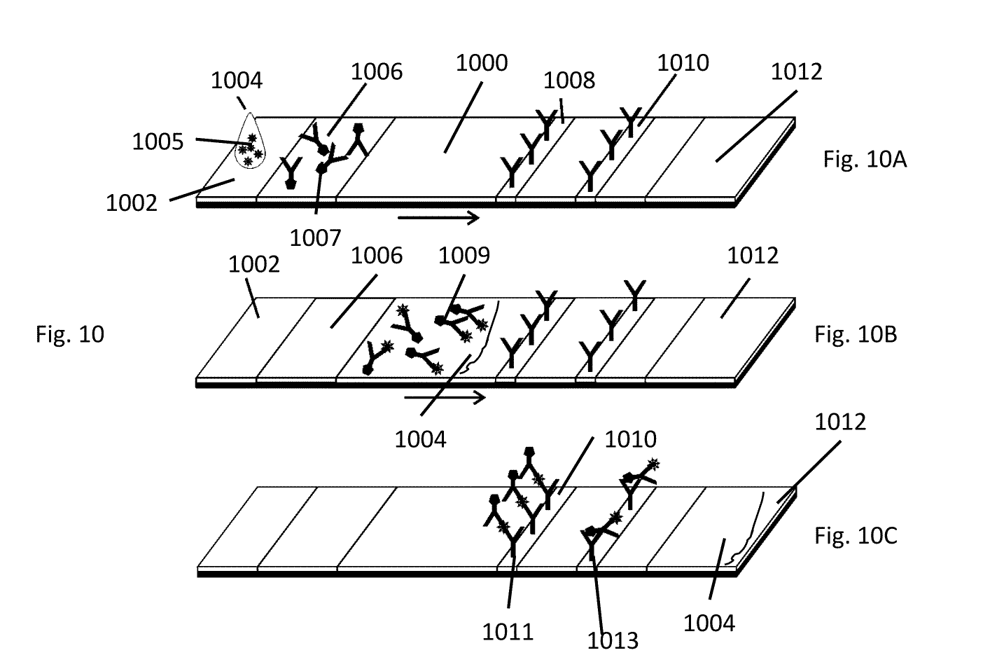

FIGS. 10A-10C schematically illustrates a typical lateral flow assay. These figures illustrate detection with colloidal gold labels. The lateral flow assays of the invention can in some cases use gold labels. In preferred embodiments, the lateral flow assays utilize fluorescent detection.

In FIG. 10A, a sample fluid, which may be a fluid sample 1004 which may be a clinical sample fluid containing target analyte 1005 is be applied to a sample pad 1002 which may be partly overlapping the membrane or substrate 1000, which may be a nitrocellulose membrane. A conjugate region comprising a conjugate pad 1006 may have gold labeled antibodies specific to the target analyte 1007 deposited thereon, wherein the gold labeled antibodies specific to the target analyte 1007 is either very loosely bound or unbound such that gold labeled antibodies specific to the target analyte interacts with the target analyte 1005 and is carried by the movement of the sample fluid 1004 which may be a clinical sample fluid by capillary action through the substrate or membrane 1000. A test region 1008 comprising antibodies specific to the target analyte as described herein is bound to the membrane or substrate 1000 positioned after the conjugate pad 1006 so that the target analyte 1005 interacts with the gold labeled antibodies specific to the target analyte 1007 prior to interacting with the antibodies bound at the test region 1008. A control region 1010 comprising antibodies specific to the Fc region of the labeling antibody as described hereinabove is bound to the membrane or substrate 1000 positioned such that the sample target analyte 1005 will interact with the test region 1008 prior to interacting with the control line 1010. A wicking pad 1012 is provided, which is adjacent to or overlapping part of the membrane or substrate 1000, and may provide a volume to enable substantially all of the fluid sample 1004 which may be a clinical sample fluid containing target analyte 1005 to pass by and interact with the test region 1008.

In FIG. 10B the sample fluid 1004 which may be a clinical sample fluid containing target analyte 1005 has been drawn by capillary action from the sample pad 1002 to and through the conjugate pad 1006 towards the wicking pad 1012, allowing target analyte 1005 to interact and bind with the gold labeled antibodies specific to the target analyte 1007 to form labeled target complexes 1009, with flow in the direction of the arrows pointing from right to left).

In FIG. 10C the fluid sample 1004 which may be a clinical sample has been drawn by capillary action into the wicking pad 1012, allowing labeled target complexes to interact with the test region 1008 and to form bound labeled target complexes 1011, wherein both the gold labeled antibodies specific to the target analyte 1007 and the bound antibodies specific to the target are bound to the target, forming a classic sandwich assay.

Any unbound target complex that passes by the test region 1008 and any gold labeled antibodies specific to the target analyte which has not been bound to target analyte interact with antibodies specific to the Fc region of the labeling antibody bound to the control region 1010.

The term “surface analyte binder” refers to the molecule bound to the lateral flow substrate or membrane which binds to the analyte of interest. This surface analyte can be one or more antibodies comprising one or more antibody types, one or more monoclonal antibodies, one or more aptamers, one or more hybridizing nucleic acids or other analyte binding moieties. These are also referred to as target or capture antibodies.

The term “membrane detection length” refers to the dimension in the direction of flow of analyte in the region where the analyte is intended to bind.

The term “membrane detection thickness” is defined as the dimension nominally perpendicular to the direction of the analyte flow which is the thinnest dimension.

The term “membrane detection width” is defined as the dimension nominally perpendicular to the direction of the analyte flow which is not the thickness dimension.

The term “lateral flow substrate” refers to the material through which analyte can be drawn by capillary action and to which surface analyte binders are bound in the detection zone.

The term “binding region” refers to a region where an analyte may be bound to a surface analyte binder. There can be multiple binding regions on a test strip.

The term “printing” refers to the application of a liquid or solid in a controlled manner where the zone of application is controlled. It includes ink-jet style printing, contact printing, piezo droplet printing, screen printing, flexographic printing, transfer printing, silk screening, spray printing, and any other form of applying a liquid or solid surface analyte binder to a membrane so that the surface analyte binder can bind to the membrane.

The term “leading edge” refers to the first portion of the binding region that the analyte flow can interact with.

The term eluent and eluent fluid and elution reagent are used interchangeably herein.

The term conjugate region refers to a region of the strip wherein the detection antibody is deposited, and is released into the eluent as it passes through the strip. In some cases, the conjugate region is a separate pad. In some cases, the conjugate region does not constitute a separate pad.

The terms upstream and downstream are used in referring to the lateral flow assay strips to refer to the relative positions of regions on the strip. The sample and optional elution reagent are added at one end of the strip (the upstream end) and flow downstream to the other end of the strip (the downstream end).

Although utilization of a lateral flow assay may reference usage for a diagnostic or clinical application, any such lateral flow assay can be utilized for any purpose, such as environmental testing, reagent purity testing, and many other applications. Although binding moieties are routinely referred to herein as antibodies, the binding moieties can be of any other type of binding moiety, such as an aptamer, a, natural or synthetic nucleic acid, or any other appropriate binding moiety.

Lateral flow assays routinely utilize nitrocellulose membranes to which capture moieties, which can be antibodies, can be nonspecifically bound, and which can be bound in specific locations on a membrane; labels which can be bound to antibodies specific to target moieties can be provided, which can be utilized together to thus create a sandwich assay. Nitrocellulose is an inherently hydrophobic material, through which an aqueous fluid readily migrates if an appropriate set of surfactants are added, allowing interactions of targets within the aqueous fluid and any capture moieties which are bound to the surface of the nitrocellulose. Typical labels include gold nanoparticles, which are bound to an antibody, which is bound to a target moiety, which can be bound to capture moieties, which can be further bound to specific locations on a membrane. Localized binding of labels observed in specific locations can thus be an indication of the presence of a target moiety in a sample. Capture moieties are typically be applied by systems which contact the membrane, or noncontact systems which apply capture moieties as droplets or streams of fluid; the capture moieties are typically be applied as a strip or line across the membrane.

In some embodiments, membranes are nitrocellulose membranes, polyvinylidene fluoride membranes, charge modified nylon membranes, polyethersulfone membranes, glass membranes, cellulose membranes, cellulose acetate, or any other appropriate membrane material.

In some embodiments it is desirable to apply antibodies or other binding moieties utilizing printing methods, which include ink jet printing, contact printing, piezo droplet printing, printing utilizing a syringe pump, screen printing, or any other compatible printing method. In some embodiments it is desirable to apply binding moieties utilizing multiple applications to the same binding region so as to allow binding of binding moieties to the membrane in a thinner layer, mitigate evaporation effects, and permit the application of binding moieties in different concentration in different regions.

In some embodiments as described hereinafter, it is desirable to apply binding moieties in a thin layer, on for example, the top surface of a membrane. If a large quantity of reagent is applied at once, the reagent may be drawn into the membrane, and may thus permit binding of binding moieties to the membrane throughout a greater thickness than desired, which may permit binding of binding moieties throughout the complete thickness of a membrane. Thus in some embodiments it is desirable to apply reagents utilizing sufficiently small volumes so as to prevent the reagent from being drawn into the membrane by more than thirty microns, or by between twenty and thirty microns, or between ten and twenty microns, or between five and ten microns, or less than five microns.

In some embodiments, it is desirable to utilize different concentrations of binding moieties in different regions, for example where a high sensitivity is desired, it may be appropriate to utilize a high concentration of binding moieties in order to capture as much target as possible; in other regions wherein it is desirable to minimize sensitivity so as to enable capture of a smaller portion of the target moieties, and thus extend the dynamic range. The different concentrations of binding moieties bound to a membrane can be effectuated by applying different concentrations of binding moieties in different regions, by applying binding moieties utilizing differing numbers of applications of applications of the different binding moieties, by applying binding moieties and applying a buffer or other reagent with either a lower concentration of binding moieties or a reagent with essentially no binding moieties, so that the binding moieties may be diluted and may thus be drawn deeper into a membrane, thus increasing the volume into which the binding moieties may be bound without significantly changing the surface area over which the binding moieties may be bound. In embodiments wherein the binding time is less than the diffusion time, a reagent with either a lower concentration of binding moieties or with essentially no binding moieties may be applied prior to a reagent with a higher concentration of binding moieties, as the binding moieties may have sufficient time to redistribute within the wetted volume prior to binding to the membrane. More than two applications of binding moieties and reagents with a lower concentration of binding moieties or with essentially no binding moieties can be utilized.

In some embodiments, it is desirable to apply capture moieties in shapes other than strips. The capture moiety may be a relative expensive reagent, and thus it may be desirable to utilize as little of it as possible; similarly, devices utilized to apply capture moieties may be relatively expensive, and it may be desirable to minimize the time needed to apply capture moieties.

In some embodiments it may be desirable to utilize a single lateral flow membrane to perform tests for multiple antigens.

In some embodiments, tests which may require lower sensitivity may have labels applied or positioned at one or more positions on the strip, positioned after the binding regions associated with tests requiring greater sensitivity. In other embodiments labels for tests which may require lower sensitivity may have labels brought in from one or more regions which may be positioned to one side or the other with respect to the main flow wherein the binding regions may be positioned, and may interact with sample antigens after the sample antigens have passed by the binding regions associated with test which may require greater levels of sensitivity.

In some embodiments, an additional wash step may be utilized to reduce background, wherein a specified volume of a wash fluid may be added to the sample pad after a predetermined period of time has passed. In other embodiments, sample fluid may be added to one portion of the sample pad, and a wash fluid may be added to a different region of the sample pad which may interact with a different fluid pathway as a part of a membrane, wherein the fluidic path to one or more binding regions may be longer, and may thus result in wash fluid arriving at the one or more binding regions after the sample may have arrived and interacted with the one or more binding regions, allowing the wash fluid to remove nonspecifically bound antigen, providing a lower background signal.

In some embodiments, quantitation of one or more sample components may be desirable. In some embodiments, the software may perform a relative quantitation of two or more targets, where at least two of the two or more targets may be present in the raw sample. In other embodiments wherein one of the targets may be a control added to the raw sample, an absolute quantitation of one or more targets may be performed by the software.

In some embodiments, calibration regions may be provided. Calibration regions may include regions which may have known quantities of analyte, to allow absolute or relative quantitation. Calibration regions may include printed regions with known quantities of analyte to verify correct operation of the system. For example, if batteries are low, optics are scratched, dirty or otherwise degraded, than a drop in the calibration region signal may be detected and the operator may be alerted, and data stored may include warnings, which may include information as to significantly the calibration region signal has degraded. Calibration region(s) may include binding region(s) to facilitate quantification of sample amount(s). For example, blood albumin can be detected to provide a check on the amount sample applied.

In some embodiments, a lateral flow strip substrate or membrane may be kept wet when it is being read. This may reduce reflection(s) and or increase fluorescence received by the detector. In some embodiments, this may be effectuated by reducing air flow over the substrate or membrane through use of a bag, pouch, cover or other enclosure to minimize evaporation. In other embodiments this may be effectuated by a reservoir of fluid, which may be contained in a housing which may hold a lateral flow test strip.

In some embodiments, a lateral flow strip may be wet with a fluid with an index of refraction greater than water, such as an index of refraction of 1.40, 1.45, 1.50, or 1.55. An index close to that of the substrate or membrane (1.50 for nitrocellulose) may reduce scattering, allowing excitation light to penetrate farther, and emission light to exit from deeper within a substrate or membrane. Wetting solutions can include organics such as glycerin, silicon oil and propylene glycol, or aqueous solutions such as sugar solutions, salt solutions such as NaCl, MgCl₂ concentrated buffers, or miscible mixtures with indexes of refraction close to the substrate or membrane index of refraction. The index of refraction of the liquid or miscible mixture of liquids may be within 0.10, 0.05, 0.02, 0.01, or 0.005 of the index of refraction of the substrate or membrane.

### Analytes

The test strips of the invention can be used with any suitable analyte for which complexation in the sample compromises detection of the analyte. An analyte is typically a compound for which a measurement of the presence of or the amount of is desired. The analyte is typically an antigen for the detection antibody. In some cases, the analyte may be an antibody or portion of an antibody. A preferred antigen is p24, the detection of which can be important in the treatment of HIV as described in U.S. Pat. No. 5,391,479 Feb. 21, 1995 and U.S. Pat. No. 5,556,745 Sep. 19, 1996. The antigen p24 is typically measured from whole blood or serum samples from a patient. The level of p24 in the patient can be used to determine the appropriate care regimen for the patient. The use of p24 in clinical samples is described in Schüpbach, Int Arch Allergy Immunol 2003; 132:196-209, Schupbach, Journal of Medical Virology 78:1003-1010 (2006), and Schupbach, Journal of Medical Virology 65:225 (2001), which are incorporated herein by reference in their entirety for all purposes.

Other antigens include Dengue nonstructural glycoprotein (NS1) as described in US Patent Application 2013/0164743, carcinogenic embryonic antigens (CEAs) as described in U.S. Pat. No. 4,272,504 Jun. 9, 1981, plasmogen activator inhibitor as described in US Patent Application 1005/0244893, Diflilaria immitus as described in U.S. Pat. No. 4,703,001 Oct. 27, 1987, cobalimin as described in U.S. Pat. No. 4,950,612 Aug. 21, 1990, human beta₂-microglobulin, human TBG, human IgE, and human urinary albumin, as described in U.S. Pat. No. 5,073,485 Dec. 17, 1991, rheumatoid factors as described by U.S. Pat. No. 5,556,745 Sep. 17, 1996, glucose as described by U.S. Pat. No. 5,571,723 Nov. 5, 1996, and phenytoin and lipids as described by U.S. Pat. No. 5,654,156 Aug. 5, 1997.

Nucleic acids can also be measured using lateral flow assays, and the test strips of the invention. For example, Nucleic acids can be captured on lateral flow test strips either in an antibody-dependent or antibody independent manner. Antibody-dependent format also called “nucleic acid lateral flow immunoassay (NALFIA)” employs an antibody capture line and a labeled amplicon or oligonucleotide probe of complementary sequence to the amplicon.

### Detection Antibodies

Detection antibodies are well known and ubiquitous in the lateral flow assays described herein and in the reference incorporated by reference. Detection antibodies are selected to bind highly selectively to the analyte of interest. The detection antibodies are labeled, again as described in detail in the references incorporated herein. A typical label is colloidal gold. Fluorescent labels are particularly useful for the test strips and methods described herein. In some cases, an antibody utilized to bind a label to a target may be modified so that the surface charge of the antibody may be reduced so as to prevent nonspecific binding to a membrane surface.

### Extended Dynamic Range and Improved Sensitivity

It is sometimes desirable to have an extended dynamic range. A typical lateral flow assay device may have a dynamic range which may be little over an order of magnitude, being limited by the size of the labels, the contrast each label may provide, and the distance or length of the binding region, which may typically be only one to two millimeters in the direction of fluid flow (y-axis), which may limit the number of labels which may be captured and bound. If all the available capture sites resulting from bound capture moieties are occupied, any unbound target may pass by the capture region and be lost.

Thus in some embodiments, where a large dynamic range is desirable, particularly in getting quantitative data at the high concentrations of analyte, we have found that it can be useful to have a longer length capture pad in the direction of the reagent flow. We have found that in order to improve dynamic range, the length of the capture or target area in the direction of the fluid flow is be greater than two millimeters, greater than four millimeters, greater than 8 millimeters, greater than a centimeter, or greater than two centimeters. As used herein capture, target, and binding are all used to refer to the region of the lateral flow assay strip where the capture antibody, or other capture moiety resides, for example to bind to the analyte-detection antibody complex for detection. Such a region may be referred to as a test region, a test line, a test stripe, a capture region, a capture line, a capture stripe, a binding region, a binding site, a binding line, or a binding stripe.

In some cases, by making the capture region relatively long, this also creates a capture area that has a relatively large area, which can be undesirable for example due to the cost of capture reagent to cover this area. Thus, we have found that a capture or target area that is relatively long in the direction of the fluid flow can be used to minimize cost, and to permit space which might otherwise be used for a single large capture moiety region to be utilized for several capture moiety regions. In some cases, the capture region has a length in the y direction over the length in the x direction of greater than 2:1, greater than 3:1, greater than 4:1, greater than 5:1 or greater than 10:1. The shape of the elongated capture region can be any shape including rectangular, elliptical, or other. Fluid flow in a lateral flow membrane is generally a laminar flow as opposed to being a turbulent flow. As a result the width required for large dynamic range and associated quantitation may be little more than that needed for spotting equipment. Thus we have found that often the width needed to provide extended dynamic range may be considerably less than the full width of the lateral flow membrane. In some embodiments, the width in the x-axis may be half the width again in x-axis of the lateral flow membrane, or may be less than four millimeters in width, less than two millimeters in width, less than one millimeter in width, or less than 0.5 millimeters in width.

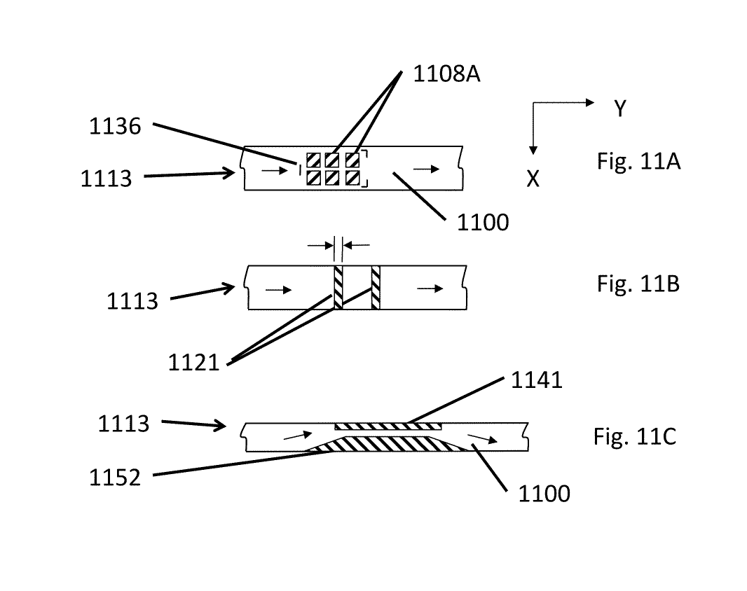

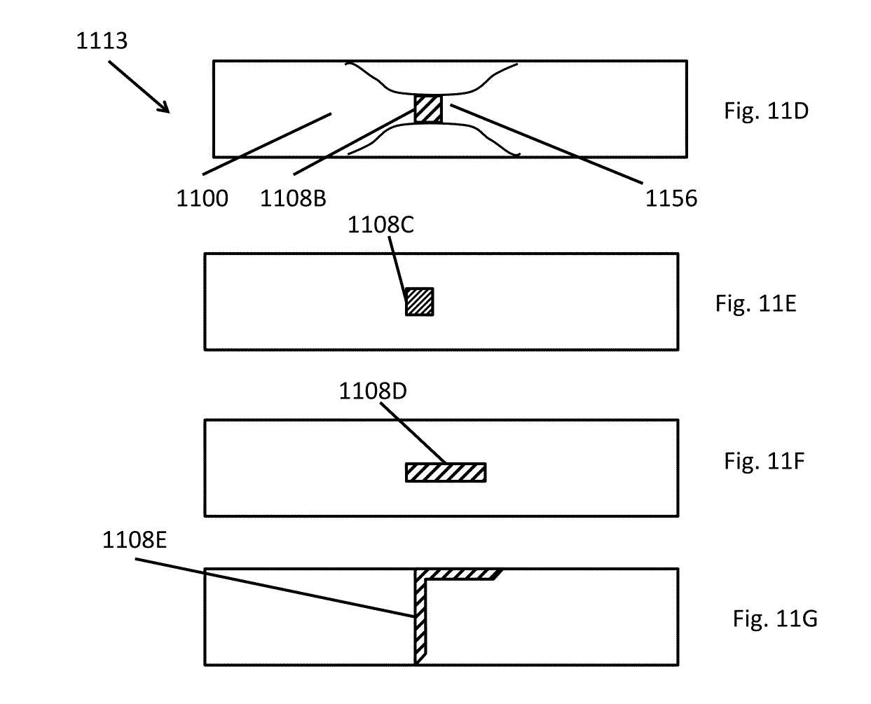

In some embodiments, the binding regions may not extend across the full width of the membrane detection width as shown in binding region 1108C in 11E. In some embodiments the length to width ratio of the binding region may be <0.2, <0.4, <0.6, <1.0, <2.0 etc. as shown in FIG. 11E, allowing high sensitivity with minimal use of expensive binding antibody. Longer binding areas may enable a greater dynamic range by allowing more surface area for target binding. Using a region such as test region 1108D that does not extend across the membrane detection width may reduce the amount of expensive labeled antibody required and may enable more tests to be done on a single strip. FIG. 11F illustrates a binding region that is relatively long in the direction of flow (y direction). As described above, a long thin binding test region 1108D, as shown in FIG. 11F, may allow for greater dynamic range.

FIG. 11G shows how the shape of the binding region can be used for both high sensitivity and high dynamic range. The test region 1108E, as shown in FIG. 11G, contains two portions, one portion with that is long in the x direction and short in the y direction for sensitivity such that all sample target analyte has an opportunity to interact with a test region, and a second portion that is long in the y direction and narrow in the x direction for providing high dynamic range. The capture region shown is in the shape of an L, but the shape of the region can be any shape, including a shape more like a T.

For an assay that has good binding kinetics and a small amount of sample target analyte, most of the target analyte tends to be bound at the leading edge of a test region. For example, using a standard test strip with a one millimeter wide striped test region, most sample may be bound within the first 100-200 microns, and any sample target analyte bound beyond that distance may be unmeasurable, so effectively no sensitivity is lost by utilizing a narrower test region, while expensive binding antibodies need not be wasted. It may be desirable to capture target analyte across the entire width of a strip, or a significant fraction thereof, as any portion of the width of the strip which is not covered by a test region results in target analyte which is lost, as it has no opportunity to be bound and measured, while capturing as much target analyte as possible allows for an improved signal to noise ratio. For a wider strip, as the concentration of target analyte is increased, the width of a test region with significant amounts of bound target analyte increases, growing wider as a function of the amount of target analyte. As there is a large amount of target analyte bound, the width of the test region across the strip (perpendicular to the direction of sample fluid flow) is no longer important, as it is no longer necessary to capture as much target analyte as possible for signal to noise purposes, thus allowing a narrow test region to have a very high dynamic range, while utilizing a minimal amount of binding antibody. But dynamic range may be increased as linear function of the length (along the axis of sample fluid flow).

In some embodiments as shown in FIG. 11A, the test strip comprises multiple test regions. The test strip with multiple test regions may be desirable as it is readily produced by a standard striper, which applies a stripe across a piece of membrane material. A spotter can apply binding antibodies at any point; but the fluidic delivery of a spotter may be in discrete spots, which may have somewhat variable morphology and density of applied antibody. Thus in some embodiments, it may be desirable to utilize several separate test regions (a plurality of test regions) in order to maximize a combination of sensitivity and dynamic range with minimal variation in quantitation which may result from variable applied binding antibody density. In some cases it is desirable to have from about 4 to about 100 test regions, or from about 4 to about 50 test regions, or from about 4 to about 20 test regions. The test regions can comprise an array of test regions, for example an array of n regions by p regions where n and p are independently 2, 3, 4, 5, 6, 7, 8, 9, or 10. For example, the array of test regions can be 2 by 3, 3 by 2, 5 by 3, 3 by 5 or any other suitable combination. The shape of the test regions can be any suitable shape including square, rectangle, circle, ellipse or other arbitrary shape. The shapes of the test regions are typically all the same, but in some cases different test regions can have different sizes and shapes.

In other embodiments, multiple test regions may be utilized wherein some test regions in one or both of the axes (along and perpendicularly to the flow of a fluid sample) may be utilized for one target analyte which may need high sensitivity and or high dynamic range, while another set of separate test regions may be utilized for another target analyte which may not need high sensitivity or high dynamic range. Combinations of stripes and separate test regions may also be utilized. An L or T shaped region may be formed with a more advanced spotter which can utilize smaller fluidic volumes, for example, nanoliter to picoliter volumes, to form a set of depositions which may be relatively uniform in overall binding antibody deposition uniformity. Similarly, such a spotter may be utilized wherein simultaneous control of fluid flow and the motion of the dispensing tip in the axis along the direction of fluid sample and the axis perpendicular to the direction of fluid flow may be effectualized, allowing the advanced spotter to create a two dimensional “stripe” forming an L or T or other form as appropriate.

One aspect of the invention is a lateral flow assay strip for quantitative analysis having a capture region having both a high sensitivity portion and a high dynamic range portion wherein the high sensitivity portion has an y to x ratio of greater than less than 2:1, less than 3:1, less than 4:1, less than 5:1 or less than 10:1, and the high dynamic range portion having a y to x ratio of greater than 2:1, greater than 3:1, greater than 4:1, greater than 5:1 or greater than 10:1, where the y direction is the direction of flow on the strip.

We have also found that in some cases, it is desirable to minimize the depth to which capture moieties may be applied, for example, such that only captured labels near the surface may be observed, while labels bound on the opposing surface, or within the membrane may not be observable, despite the relative thinness of the membrane. In these cases, it is desirable to apply capture moieties with sufficiently small droplets such that the liquid is not absorbed fully into the membrane, but instead penetrates the top section, region or volume of the membrane. For example, the capture moieties are substantially present only in the top 0.01 mm, the top 0.02 mm, or the top 0.05 mm. In some cases the capture moieties are substantially present only in the top 40%, or the top 20% or the top 10%, or the top 5% of the thickness of the membrane in the capture region.

In other embodiments it is desirable to have high sensitivity, and thus it is desirable to capture as much target as possible. It may thus be desirable to apply capture moieties complete across the flow of fluid, similar to methods currently in use. It may however be desirable to additionally modify the fluid flow pathway so as to cause the fluid to flow through a smaller cross sectional capture area, thus improving the signal to background, as the background level is fixed, and the observable area may be minimized. The signal compared to the unmodified strip is increased because the same number of analyte molecules may be captured in a smaller area. The cross section of a membrane or substrate 1100 in the area of a test region 1108B may be minimized by changing the shape of the membrane, for example, cutting or grooving the membrane as shown in FIG. 11D to form a narrowed membrane region 1156, thus providing a narrowed flow path. Alternatively, the fluid flow may be modified in a narrowed membrane region 1156 so as to conform to a similar flow profile by blocking movement of fluid by the application of wax or other materials which may fill the pores of the membrane, or by locally increasing the hydrophobicity of the membrane, so as to prevent an aqueous fluid from wetting and being drawn into the areas with increased local hydrophobicity by capillary action.

Thus, one aspect of the invention is a lateral flow assay strip in which the x dimension of flow in the strip is narrowed at the capture region (test line) as compared to the width of flow for the strip preceding the capture region, for example for the portion of the strip at the sample addition region. In some cases, the dimension of flow in the capture region is 80% or less of the x dimension of the strip preceding the capture dimension. In some cases, the dimension of flow in the capture region is 60% or less of the x dimension of the strip preceding the capture dimension. In some cases, the dimension of flow in the capture region is 50% or less of the x dimension of the strip preceding the capture dimension. In some cases, the dimension of flow in the capture region is 20% or less of the x dimension of the strip preceding the capture dimension.

In some embodiments as shown in FIG. 11D, it may be desirable to utilize a shape for a membrane region which may be narrowed as described above for improved sensitivity relative to a fluid flow, and may subsequently be widened relative to a fluid flow in order to allow for detection with higher dynamic range.

In some embodiments, the binding area may be created by printing. In other embodiments the printing may be performed using multiple applications, with time between the dispensations to allow binding of the surface analyte binder to the substrate or membrane, so as to facilitate a thinner layer or surface analyte binder to be deposited in the upper region of the lateral flow substrate. This may reduce the amount of expensive surface analyte capture reagent as only the top section of the lateral flow substrate is detectable.

In some embodiments, a capture reagent may be printed in to create a uniform concentration across the membrane detection thickness as shown in FIG. 11C wherein a capture reagent is shown as being applied only to the top portion of a membrane or substrate 1100, and not throughout the thickness of the membrane or substrate 1100. In some embodiments a surface analyte binder may be printed to create a non-uniform concentration across the membrane detection thickness. In other embodiments a surface analyte binder may be applied with a gradient which may either increase or decrease in the direction of analyte flow. In other embodiments a surface analyte binder may be applied with different concentrations near the edges of the membrane detection width. This may provide a higher contrast to better facilitate binding area identification.

In some embodiments, the leading edge of a binding region may be wider than other parts of a binding region. This may enable a wider initial contact area to improve low concentration detection.

In some embodiments as shown in FIG. 11A, one or more fiducials 1136 may be provided on the lateral flow carrier, membrane or substrate 1100. A fiducial(s) 1136 may aid in determining an image area that represents the binding region(s). This may increase quantitation accuracy as it may allow more accurate collection of signal from binding region(s). For example, a trailing edge(s) may often be poorly defined. In some embodiments a fiducial(s) 1136 may be a printed, embossed, perforated, molded or otherwise recognizable feature. A fiducial(s) 1136 may be one or more fluorescent particles attached to the substrate or membrane 1100. A fiducial(s) 1136 may allow algorithmic localization of test region(s) 1108A of interest. A fiducial(s) 1136 may be used to verify correct insertion of a lateral flow device test strip 1113. In some embodiments a fiducial(s) 1136 may be created by an assay control feature. In some embodiments, a fiducial(s) 1136 may be used to verify image quality or focus, or may be utilized to permit setting of focus. In some embodiments, a fiducial(s) 1136 can be used to generate a point spread function to allow image processing to algorithmically enhance the image, including improving quantitation, dynamic range, and sensitivity of the image. In some embodiments, fiducials 1136 may be formed in the shape of lines, crosses, circles, discs, or any other shape which may be useful. In some embodiments it may be desirable to print or otherwise cause to bind fiducials to a lateral flow substrate or membrane which may comprise ink, fluorescent dyes, fluorescent particles, or a control material. A lateral flow device test strip 1113 may be referred to as a lateral flow device, a test strip, a lateral flow test strip, or a lateral flow strip.

The following describe methods to decrease the dimension of the nitrocellulose in the z-axis (thickness); i.e. to make it functionally thinner. Although the nitrocellulose is already quite thin, the molecules of analyte that occupy the interior of the nitrocellulose are lost to detection. If the analyte molecules can be limited to binding to the top surface the detection limit can be improved. In some embodiments, a lateral flow membrane or substrate 1100 may be printed on the back of the lateral flow substrate or membrane with a substance that impedes fluid flow 1152. In other embodiments, the substrate or membrane may be deformed by, for example, compressing the back of the substrate or membrane. As shown in FIG. 11C this may be used to increase the flow of the analyte into a upper portion of the membrane region thickness of a detection zone region 1141 which may typically be the top 10 um, but may be within the top 2 um, the top 2 to 5 um, within the top 5 to 10 um, within the top 10 to 20 um, within the top 20 to 40 um, or within the top 40 to 60 um of the membrane region thickness of the membrane or substrate 1100.

In some embodiments, binding regions may be utilized in shapes other than lines. Shapes can include rectangles, sections of variable width, shapes where the width (measured in direction of flow) to length (perpendicular to flow) W/L ratio is <0.2, <0.4, <0.6, <1.0, <2.0 etc. Wider areas provide greater dynamic range by allowing more options for target binding. Using areas that do not extend across the flow strip can reduce the amount of expensive labeled antibodies required.

In many lateral flow assays improved sensitivity and dynamic range may reduce the number of errors that may occur due to analyte level variations. Insufficient sensitivity can lead to false results when test fails to detect a low titer analyte. In some cases a high titer sample can result in a false negative due to the prozone or hook effect. A high and preferably linear dynamic range is especially important for assays in which quantitative data is desirable.

In some embodiments, data from multiple images utilizing the same exposure time may be combined to reduce read noise. In other embodiments, multiple images, with some taken utilizing different exposure times may be used to extend the dynamic range. In some embodiments, an exposure may be taken, analyzed, and another exposure may be taken with an exposure time determined by the previous exposure, wherein the new exposure time may be selected so as to effectuate a desired signal level for a particular region of an image particularly for a camera wherein the output of said camera may be nonlinear, for example, of a test region. In further embodiments, additional images may be taken wherein other region(s) may have different levels or values of label, and different exposure times may be useful so as to allow more accurate quantification of said labels in said different regions. In some embodiments one or more images may be taken utilizing short exposure times to prevent any part of the images corresponding to the binding region(s) of the detector from saturating. The images may be analyzed and if the signal is not saturated, a longer exposure time may be used to improve the signal/noise while avoiding detector saturation. One or more additional images may be taken utilizing longer exposure times, wherein a portion of the images corresponding to the binding region(s) of the detector may be saturated, and the short exposures and the longer exposures may be combined, wherein any portion of the longer exposure which is saturated may utilize data from the short exposure, multiplied by the ratio between the exposure times.

In lateral flow assays utilized with a high concentration of analyte, surface analyte binders in the leading edge may become fully loaded. Unbound analyte will continue to flow until the unbound analyte reaches unbound surface analyte binders in the binding region. In some embodiments all surface analyte binders in a binding region may be bound to analyte. For low concentration samples most of the sample analyte may bind at the leading edge of a binding region. In other embodiments only a portion of the binding area, such as the leading portion of a binding region, may be used in order to improve detection of a low concentration analyte.

In some embodiments, the background associated with images may not be zero due to a combination of native fluorescence, non specific binding, dark current, camera offset levels, light leakage, etc; the background level can be determined from regions outside of binding regions. Multiple data points may be combined to establish the intrinsic background which may be subtracted from the total signal to generate the signal of the target.

In some embodiments, illumination light may not be uniform, and the system may compensate for the lack of uniform illumination. A profile of an illumination pattern may be captured and the data adjusted to correct for this variation. In some embodiments an illumination pattern maybe characterized utilizing one or more calibration images. A test surface may be utilized to characterize an illumination pattern. In some embodiments a test surface may be part of a consumable associated with a lateral flow test; a test surface may be arranged so as to be on the back of a lateral flow membrane wherein there may be a impermeable and nonporous layer betwixt the test surface and the lateral flow membrane; in other embodiments, the test surface may be packaged with a lateral flow membrane, but may not be directly affixed thereto; in further embodiments, the test surface may be positioned, for example, wherein the test surface may be visible from the same side wherein the lateral flow membrane may be imaged, wherein a cartridge which may hold both the test surface and the lateral flow membrane may be rotatable, and by rotating the cartridge by 180 degrees, either the lateral flow membrane or the test surface may be imaged. In some embodiments a test surface may be provided as a separate calibration tool. In some cases an illumination pattern may be characterized utilizing a surface with a uniform fluorescence. A characterized illumination pattern may be used to verify appropriate functionality of the optics, or to permit relative or absolute quantitation of different binding regions. An exposure time may be set to generate an appropriate calibration image, and multiple images may be used to reduce noise from the calibration image(s). The illumination pattern may be smoothed using algorithms known in the art.

In some embodiments, one or more dark images wherein the excitation light may be inactive may be captured for calibration. This image may be utilized to identify hot pixels to be excluded from analysis, and to determine dark current or light leakage.

In some embodiments, a background subtraction may be effectuated by the use of an algorithm which utilizes a rolling ball or sliding paraboloid algorithm; in further embodiments, a deconvolution method may be used to find and discard dust particles and or to better fit a region of interest than may be possible using a more standard three sigma above background approach, which may also be effectuated to determine a signal level for a region. A deconvolution approach may be an iterative approach, wherein the shape of a form to be deconvolved may be varied as determined by a subsequent deconvolution to better fit the particular shape of a region, which may be variable as a result of binding levels and region morphology.

The novel test strips described above are particularly useful for quantitative analyses on lateral flow test strips, for example, using the fluorescent strips, methods, and systems described herein.

### Controlling the Shape of Fluid Flow

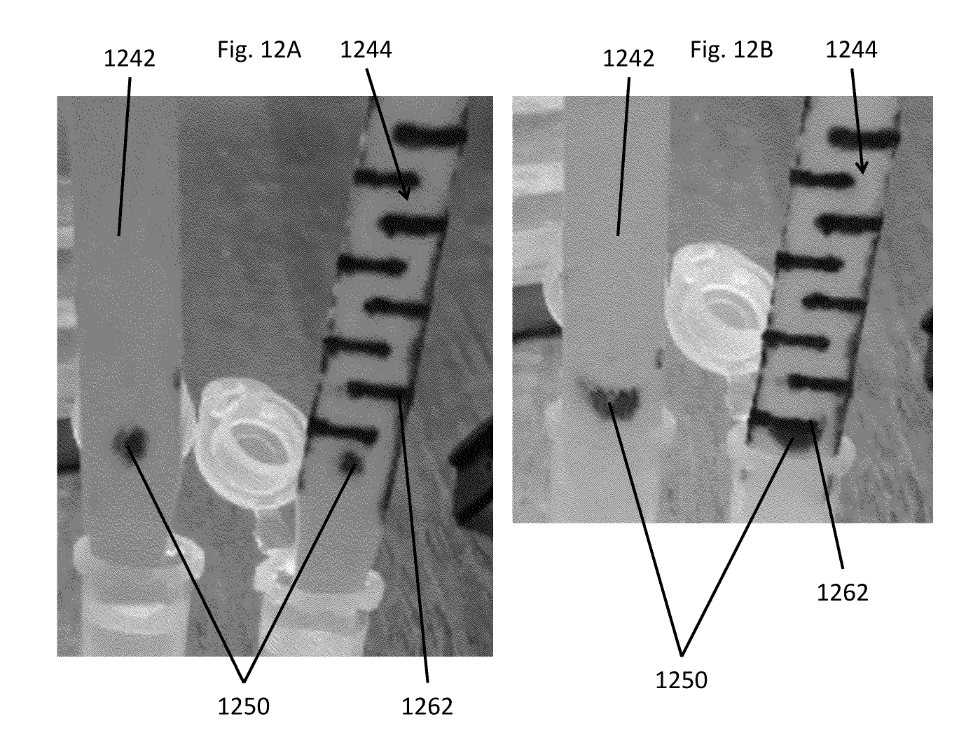

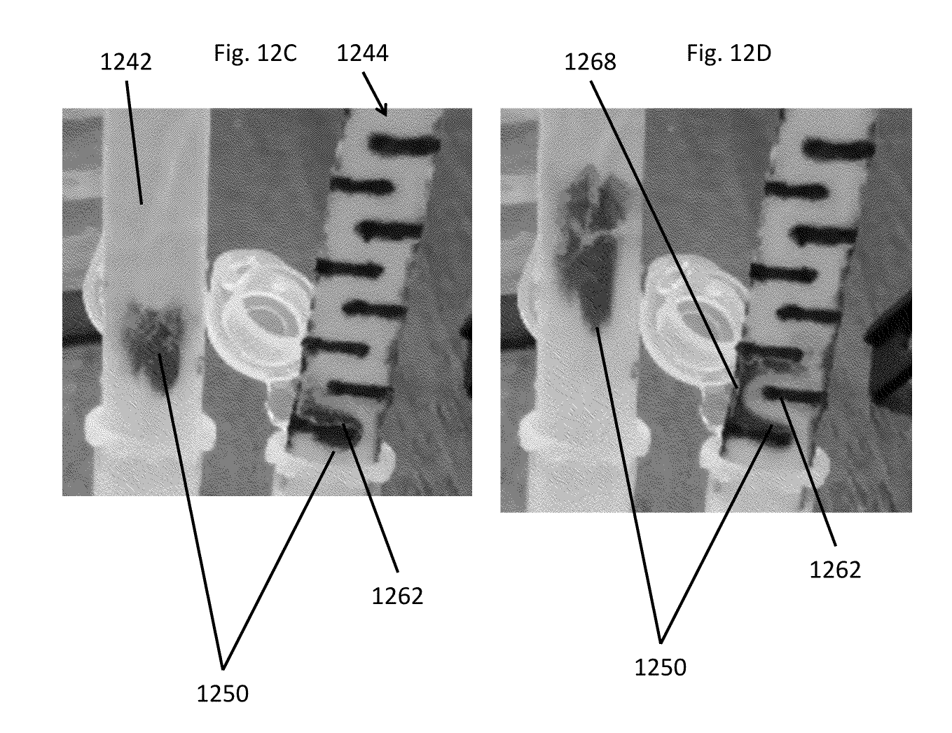

In some embodiments as shown in FIGS. 12A-D, we have found that the performance or the lateral flow assay can be improved by altering the shape of fluid flow in a membrane such that flow may induced to preferentially flow in some regions relative to other regions. FIG. 12A illustrates a set of exemplary membranes, one a membrane without fluid flow shaping 1242, and one a membrane with fluid flow shaping 1244 wherein fluid flow shaping is effectualized by a hydrophobic barrier layer such as a wax layer 1262 made apparent by subsequent markings as an interdigitated set of lines. Visual indicators 1250 were spotted onto both a membrane without fluid flow shaping 1242, and onto a membrane with fluid flow shaping 1244. Both membranes were immersed into a buffer solution in different respective vials.

Interactions of the buffer with surfactant with the hydrophobic membranes 1242, 1244, induce fluid flow as seen by movement of the visual indicators 1250 in FIG. 12B. Fluid flow in membrane without fluid flow shaping 1242 is laminar, although diffusional effects cause widening of the visual indicators 1250. The visual indicator in the membrane with fluid flow shaping 1244 can be seen to begin to flow around the wax layer 1262.

In FIG. 12C, fluid flow in the membrane without fluid flow shaping 1242 continues to move upward, while the fluid flow in the membrane with fluid flow shaping 1244 has wrapped completely around the first interdigitated portion of the wax layer 1262. In FIG. 12D, the visual indicator 1250 in the membrane without fluid flow shaping 1242 can be seen to have advance significantly further up the in comparison with the movement of the visual indicator 1250 in the membrane with fluid flow shaping 1244. The first interdigitated wax layer 1262 can be seen to be significantly wider than the marking which was applied after application of the interdigitated wax layer 1262, and produces a region with highly restricted flow 1268 through which all fluid flowing upward must pass. In some cases it may be desirable to restrict the flow at the edge by wax or other flow inhibiting methods to improve the performance of the interdigitated region.

The hydrophobicity of the membrane can be controlled such that the flow rate of the fluid can provide sufficient time for interaction and binding of substantially all target moieties passing through a binding region in a highly restricted flow region 1268, thus concentrating target moieties and labels into a small region, allowing higher signal to noise and higher signal to background ratios.

In some cases, binding regions with high dynamic range as described above may be desired. For example, a high dynamic range binding region may be placed in a region wherein flow may be shaped so as to have a minimal flow relative to other portions of the membrane with fluid flow shaping.

In some embodiments, it is desirable to prevent nonspecific binding of labels, for example, labeled antibodies, to the membrane surface. Thus in some embodiments, it is desirable to apply a coating to the surface of the membrane, for example, applying the coating after application of binding moieties. A coating may comprise at least in part, Polyethylene glycol (PEG), various proteins such as BSA, casein, surfactants such as Tween® 20, and various other proteins and surfactants.

In some embodiments, calibration areas may be printed or otherwise associated with a lateral flow membrane or substrate. Calibration may include known intensity zones to allow absolute quantitation. Calibration zones can include binding area to facilitate quantitation of sample amount. For example a common component of the sample such as blood may utilize blood albumin to determine the amount of blood loaded and utilized in the assay.

The lateral flow membrane or substrate may be bagged, or enclosed during analysis such that evaporation is minimized so as to retain fluid to reduce reflection or enhance fluorescence. In an alternative embodiment, a nonaqueous fluid or a miscible mixture of an aqueous and nonaqueous fluid may be utilized such that evaporation is reduced relative to an aqueous fluid, so that the fluid may evaporates sufficiently slowly as to remain appropriately wetted during the time needed for an assay to be performed.

The membrane may be wetted with a fluid that has an index close to the index of the wicking substrate so as to minimize scattering of excitation light so as to allow the excitation light to penetrate further into the lateral flow membrane, and to allow emission light to better exit without scattering from deeper in membrane. In some embodiments, wetting solutions may include organics such as glycerine, silicon oil and propylene glycol or aqueous solutions such as sugar solutions, salt solutions such as NaCl, MgCl₂ concentrated buffers, or miscible mixtures with indexes of refraction approaching the wicking substrate index of refraction.

### Decreasing the Prozone Effect

The prozone or hook effect results from having a high concentration of antigen relative to the concentrations of labels and bound antibodies as may occur in assays of malaria and syphilis. As a result of these concentrations, a small percentage of the antigen is bound with labels; a small percentage of the antigen is bound by the bound antibodies. One might expect a linear reduction in signal as the antigen concentration rises; this is typically not seen, particularly with gold labels as the unlabeled antigen is more mobile than the labeled antigen, and thus outcompetes the labeled antigen for binding sites on the surface. Thus as the number of antigens rises in a sample, the amount of bound label drops steeply, and may be unobservable at concentrations only slightly above concentrations which give maximum signal.

In some embodiments, prozone or hook effect may be at least partly mitigated by either providing more labels, or providing more antibodies bound to membrane, or both. In further embodiments, the label provided may not substantially change the diffusional speed of the desired antigen. The label may, for example, be a fluorescent label instead of a gold, carbon, or latex nanoparticle.

In other embodiments, multiple capture regions with antibodies which may bind to the desired antigen may be utilized, wherein different flows are utilized for the different regions such that one region may be significantly diluted relative to at least one other region, allowing a substantial percentage of the antigen to be bound to the bound antibodies in the region wherein a diluted portion of the sample is caused to flow.

In further embodiments, the sample antigens may be allowed to interact with the bound antibodies in one or more binding regions prior to interacting with labels. Thus if there are more antigens than binding sites, excess antigen will pass by the binding region, but the binding region will be saturated with bound antigen; the labels may then be introduced and permitted to interact with any bound antigens, which if the binding site is saturated with antigens, will be essentially all of the sites; the labels may then bind to essentially all of, or a significant fraction of the bound antigens, and may give a large signal. A sample which has even more may provide marginally more signal as a result of even more fully saturating the binding regions binding sites provided by bound antibodies, and may thus provide slightly more signal, as opposed to a significantly reduced signal as would otherwise occur due to the Prozone or hook effect.

One aspect of the invention provides for reducing or eliminating the prozone effect by allowing the analyte to reach the target or capture pad before allowing the detection antibody to reach the target or capture pad. In a typical lateral flow assay, the analyte and eluent pass through the conjugate region, solubilizing the detection antibody. We have discovered that the prozone effect can be reduced or eliminated by either having the analyte bypass the conjugate pad, or by adding the detection antibody to the strip in a separate addition step.

Having the analyte bypass the conjugate pad containing the detection antibody can be done in several ways. One is to have two lanes at the start of the strip—one for the analyte, and the other for eluent to pass to bring up the detection antibody. It is desired not just that the detection antibody be in the other lane, but that the detection antibody reach the capture pad later. This can be accomplished by using separate additions of sample and eluent, with a later addition to the lane with the detection antibody. We have also found that a two lane solution can be implemented in which there is only one addition of eluent to the strip. This is accomplished by having the lane in which the detection antibody travels move slower than the lane containing the antibody. We have described above how the path-length of a strip can be increased, for example by creating a serpentine pathway on that lane of the strip. This longer pathway can be used to slow the travel of the lane in which the detection antibody is traveling, or to increase the path length over which the detection antibody travels, slowing the time of delivery of said detection antibody relative to the arrival of a target analyte to a test region.

For example, a lateral flow assay test strip for a reduced Prozone effect can have an elution reagent addition region, and then following the elution reagent addition region, the strip has a portion with two parallel lanes. One lane is referred to as the sample lane, where the sample is added, and the other lane is referred to as the conjugate lane with a conjugate region having a deposited detection antibody. Sample is added to the sample application region, then elution reagent is added to the elution reagent addition region located upstream of the two lanes. Portions of the flow of the elution reagent flow into each of the two lanes.

The elution reagent flows down both the sample lane and the conjugate lane, and the test strip is configured such that the rate of travel down the strip for the detection antibody in the conjugate lane is slower than the rate of travel down the strip for the sample in the sample lane, such that the sample reaches the test strip before the detection antibody reaches the test strip. This type of test strip allows for one addition of elution reagent to result in a different relative rate of travel of flow for the two different components. Here, the time lag between the arrival of the sample and the arrival of the detection antibody is controlled by the structure of the test strip, and is not substantially dependent on the relative timing of the addition of sample and addition of reagent. Slowing the rate of travel in the conjugate lane can be done, for example, by increasing the path length. Methods of doing this are known in the art, including the methods described herein, such as by creating a serpentine path. The path length can be changed, for example by printing hydrophobic portions which direct the flow from side to side, for example, by printing interdigitated lines.

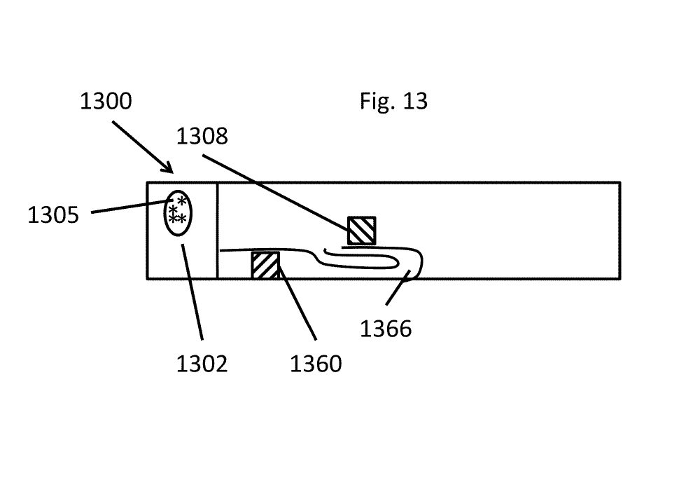

In further embodiments, labels, which may be allowed to interact with antigens after binding in binding regions with bound antibodies, may be applied to a sample pad after the sample has been applied to the sample pad, and may be applied as part of a separate pipetting step. In other embodiments as shown in FIG. 13, the labels 1360, which may be applied as a part of a manufacturing process, and may have been applied to a conjugate lane, may be allowed to interact with any sample antigen target analyte(s) 1305 after the sample antigen target analyte(s) 1305 have been bound in test regions 1308 to binding antibodies bound to a membrane or substrate 1300, as a result of being in a conjugate path or region with separate longer path reagent flow 1366, which may have a longer fluidic path length than a sample path. The labels may further have a lower R_{f} (flow resistance), allowing the labels 1360 to flow to and interact with any bound antigen in the binding region 1308 prior to interacting with any antigen target analyte 1305 which may be applied to the sample pad 1302 and may thus flow and interact with bound antigen in the binding region, rather than binding to unbound antigen and thus being unavailable to bind with bound antigen. In other embodiments the R_{f} of the antigen target analyte 1305 may be less than the R_{f} of the labels 1360, but the distance between the location of any applied labels 1360 to the binding region 1308 may be sufficiently short relative to the distance any antigen applied to the sample pad 1302 may need to travel in reaching the label 1360, that the antigen target analyte 1305 may not catch up to the label 1360 prior to the label 1360 reaching the binding region 1308 and interacting with any bound antigen, thus allowing a number of labels 1360, which may be significantly lower than the number of antigens, to effectively label bound antigens, and produce a signal. A region with separate longer path reagent flow 1366 may be generated by utilizing a wax barrier, by slitting the membrane, or by any other appropriate method.

### Methods

Aspects of the invention comprise methods for detecting and for measuring levels of analytes in samples using the test strips described herein. Those of skill in the art will understand from the descriptions of the lateral flow test strips how they can be used in methods of measuring analytes.

For example, in some aspects, the invention provides a method for detecting an analyte, which analyte may comprise analyte-antibody complexes in a sample. To carry out the method, a test strip is provided, the test strip having a sample application region for adding the sample, and in some cases also an elution reagent addition region in order to add eluent to facilitate flow. In order to provide decomplexation of the complexed antigen, the strip has a decomplexation region that acts to dissociate any complexes such as analyte-antibody complexes in the sample. In some cases, the strip also has a neutralization region in order to ensure that the environment is not dissociating when the sample reaches the conjugate region. The decomplexed analyte in the sample passes a conjugate region comprising a detection antibody or other labeled detection moiety that selectively associates with the analyte. The sample then continues through a flow region, then passes through a test line comprising immobilized test antibody or other immobilized moiety which may bind the target analyte. The test antibody will bind to analyte, and analyte that is bound to detection antibodies will be detected, for example, by fluorescence. This method allows for improved detection of analytes, which may be complexed in the sample in which they reside. A flow region may be a portion of a membrane or substrate between a conjugation region and a test region, which may allow for additional complexation between a detection antibody or detection moiety and a target analyte relative to a system with\out a flow region between a conjugation region and a test region, thus improving assay sensitivity.

Another aspect of the invention is a method for measuring both decomplexed and complexed analyte levels in a sample. The method involves having a test strip that has two separate lanes. The first lane has a decomplexation region for dissociating analyte-antibody complexes in the sample, and the second lane does not have such a decomplexation region. This allows for measurement of both decomplexed and undecomplexed analyte on the same strip. Each lane has a conjugate region comprising a detection antibody that selectively associates with the analyte, a flow region, and a test line. Measuring signal corresponding to the detection antibody at both the first lane test line and at the second lane test line allows the user to determine both decomplexed and complexed analyte levels in a sample on the same strip.

### Illumination and Imaging System

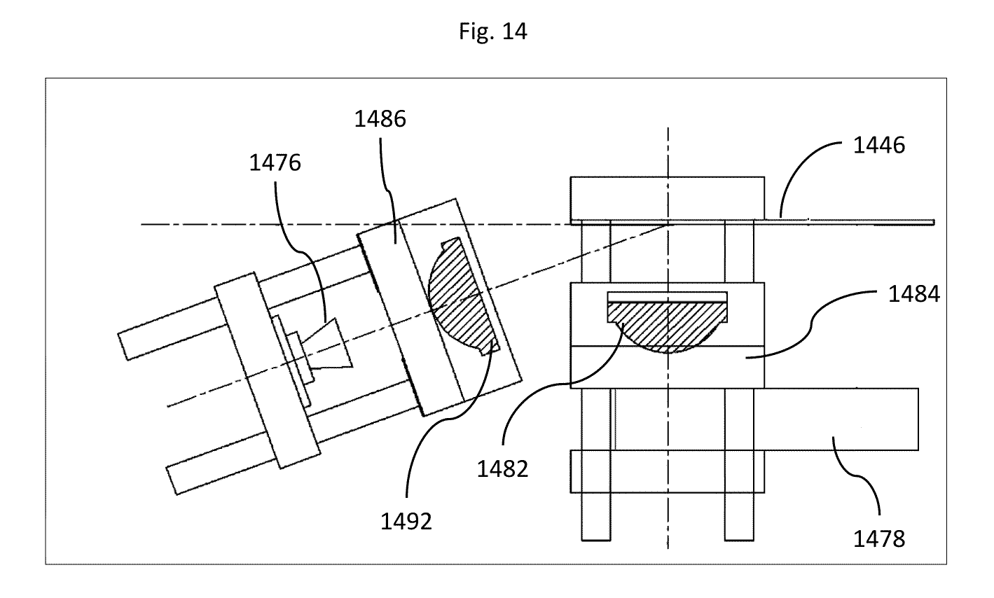

In some embodiments as illustrated in FIG. 14, an off axis illumination system may be utilized. Such a system may minimize backscatter collected by the collection lens while eliminating the need for an expensive dichroic beamsplitter.

In some embodiments, a flash system which may be a part of the camera may be utilized as an excitation source for either an absorptive or fluorescence assay.

In some embodiments, LED illumination may be utilized. High power LEDs costs have significantly dropped, while the number of wavelengths available has significantly increased. In further embodiments, a diffusing element may be utilized to provide for more uniform illumination. The diffusing elements may be a ground glass, a diffuser, a sapphire diffuser, a plastic diffusing element which may be ground or molded or may be any other appropriate diffusing element. The diffusing element may be formed as part of a lens or excitation filter. In some embodiments, a lens in the either the excitation or emission path may also perform as a filter. The lens may have a filter material bonded or affixed to the lens, or the lens may be formed from a colored glass or plastic filter material. In further embodiments, a lens in the excitation path may also serve the function of filtration and diffusion, wherein the lens may be formed from a filter material, and may be molded or ground so as to perform additionally as a diffuser. In other embodiments, a reflector, which may be integrated with an LED, may obviate the need for an emission lens.

In some embodiments, the LED light source(s) may be utilized in a modular format, utilizing a standard connector, mounting hardware, and pins, stops or other mechanisms for alignment. The LED source(s) may thus be made to be interchangeable so as to enable the use of different dyes. Excitation filter(s), lens(es) may be provided with the LED source(s) so as to provide a complete module. The LED light source(s) may be provided with an encoding mechanism so that the system can determine what LED source, which may include LED type, nominal LED current, which may have been determined using a calibration procedure, filter types, and lens types, is currently being utilized, and determine the suitability of a particular source for an application. The LED source may further comprise an LED driver, which may provide a visual indicator to a user that a battery power supply for the LED is sufficiently charged by illuminating, for example, a power switch. Said illumination may be steady when sufficient voltage is available, and may flash to warn that the voltage is low, and may not illuminate when the voltage is insufficient to provide sufficient current to an LED. The system may utilize different LED drive currents dependent on which LED source type is utilized, and may report the LED module type and serial number as part of the data which may be stored in association with an assay. The LED module source type and other data associated with the LED module may be stored in a memory associated with the LED module, which may be an EERAIVI, a Flash RAM, or any other appropriate memory. Access to the data associated with the memory and or to the status of the battery may utilize a wired connection using wires not utilized for powering of the LED module, which may be a serial connection such as a USB, SPI, I²C, 1-Wire® connection or any other appropriate serial hardware and software protocol. Alternatively, access to the data may be provided utilizing the wires associated with powering the LED, utilizing a wired RF link. Access to the data associated with the LED module may result from the use of a RFID chip. The system may be an active reader passive tag, a passive reader active tag, or an active reader active tag. The RFID chip with associated memory may be powered by power supplied for the LED module, or may be a passive device. The reader for the RFID chip may be a part of the fluorescence lateral assay system, or may be part of a smart phone.

In an alternative or additional embodiment, a back side illumination system may be utilized with a transparent or semitransparent substrate. This may be useful when different types of assays requiring different excitation wavelengths may be desired. The different LED modules may be activated at different times, or may be activated at the same time, or both sequential and simultaneous usage may be utilized. In some embodiments, a back side illumination system may be utilized for absorbance measurements, while an off axis illumination system may be utilized for fluorescence measurements. In a further embodiment, multiple off axis illumination modules may be utilized, and may be utilized in conjunction with a back illumination module.

In some embodiments, TIRF illumination may be utilized either in conjunction with or instead of back illumination and or off axis illumination.

In some embodiments, Fresnel lens for excitation or collection lens may be utilized so as to improve spacing requirements. An excitation Fresnel lens may further incorporate a diffusion element as part of the Fresnel molding process, so that only one side of the Fresnel element needs to be formed, while the opposing side may be a planar surface.

In some embodiments two lenses may be utilized so as to increase the optical power, allowing the lenses to be located farther away for physical access for other portions of the optical system. The two lenses may have the same optical power or may have different optical powers.

In some embodiments, a one to one magnification system may be utilized; in other embodiments, other magnification levels may be utilized; in some embodiments it may be desirable to utilize a one to one magnification system for a common camera, while other cameras may be utilized with different magnification levels which may be designed to match a particular cameras smaller or larger image sensor.

In some embodiments, wherein more image data may be desired then may fit within a single image, several images may be utilized, each of a different portion of the lateral binding region(s). The images may overlap so as to allow for complete coverage of the binding region(s), or may image separate binding regions, wherein the binding regions may have gaps therebetween so as to prevent bleaching. When bleaching is a concern, the excitation light may be configured with an aperture so as to prevent excitation light from illuminating adjacent areas. In other embodiments, wherein bleaching is not a concern, adjacent areas, which may include all or substantially all of the binding regions may be simultaneously illuminated. In some embodiments wherein communications between a camera module and an LED device, which may comprise an LED driver, power to the LED may be synchronized between the camera and the LED driver so as to extend battery life and to prevent photobleaching.

In some embodiments wherein all or substantially all of the binding regions are simultaneously illuminated, the optics may be further configured so as to allow movement of the camera relative to the collection optics so as to permit imaging of different portions of the illuminated area.

In other embodiments, the binding region(s) may be moved relative to the optical system, such that there is no movement of the collection optics and camera relative to the excitation optics so as to allow different portions of the substrate or membrane to be excited and imaged.

In some embodiments, a mechanism may be utilized which may be a sliding movement, a rotating movement or any other type of movement which effectuates the desired relative motion. In some embodiments, detents, reference alignment marks, guide pins or other means for alignment may be utilized. The means utilized may require the user to move and align the system, wherein the user may need to actuate a clamp mechanism to prevent movement, or the system may be configured such that sufficient friction is present in the mechanism so as to prevent further motion without further user action.

In other embodiments, the mechanism may utilize detents, stops of other devices so as to provide the user a clear tactile indication that the relative motion is properly aligned.

In some embodiments, the system may provide fiducials or other optical indicia which may be imaged by the smart phone camera so as to insure that the system is properly aligned. The smart phone may analyze the image(s) and provide feedback to the user as to whether an image was properly aligned, and as to whether an image was of the expected region. The indicia may be fluorescent indicia, or may be reflective or absorptive indicia, which may require the movement of an excitation or emission filter so as to allow sufficient reflection or absorption to be imaged, or the system may be configured so as to have sufficient in band light emitted from the excitation and collected by the collection system and imaged by the smart phone camera as to provide appropriate measurement of the indicia. In some embodiments the indicia may be a trademarked indicator, such that only a licensed lateral flow assay may be utilized with the device.

In some embodiments, wherein a binding region(s) may be larger than may be imaged in a single image, multiple images may be “stitched” together into a single image by a processor. Said stitching may be performed after any appropriate normalization performed by a processor for excitation and collection optics (flat fielding), and any spatial modulation needed for image distortion such as barrel distortion, pincushion distortion, or other distortion caused by imperfect optics. In some embodiments, a processor may further linearize data received from a camera which has a non-linear output, such as a camera which has a built-in gamma function or other non linear functions utilized by the camera to increase dynamic range functions intended to improve visibility in shadows.

In some embodiments, a smart phone adapter and retention mechanism may be utilized to hold a particular model of phone in position relative to system optics. Different adapter and retention mechanisms may be utilized for different models or styles of phones, compensating for thickness, width, height, curvature of case, position of camera relative to edges, position of any switches or screen which might be otherwise inadvertently activated by pressure, contact, or proximity from being mounted in the adapter and retention mechanism of a phone.

In some embodiments, a smart phone adapter and retention mechanism may be adapted such that a smart phone may be slid into the smart phone adapter and retention mechanism. In other embodiments, a smart phone adapter and retention mechanism may be hinged with to allow the smart phone adapter and retention mechanism to accommodate any phone protrusions which might prevent a phone from sliding into a smart phone adapter and retention mechanism.

In some embodiments, a light seal may be provided as part of a smart phone adapter and retention mechanism. The light seal may be configured to be between the smart phone about the smart phone's camera and the smart phone adapter and retention mechanism, or between the smart phone about the smart phone's camera and the lateral flow fluorescence system, or there may be two light seals, one between the smart phone about the smart phone's camera and the smart phone adapter and retention mechanism, and one between the smart phone adapter and the lateral flow fluorescence system. The light seal may comprise molded features which may tightly fit to a smart phone, and may require several reflections even were light to pass by a part of the seal which may be intended to seal against the smart phone and may be formed of materials such as a foam material, a felt material, or a combination of various materials as needed for a particular configuration. The light seal may be configured to substantially block ambient light from entering the smart phone camera and providing a significant and potentially variable background. The seal may be further configured to block sufficient light so as to prevent significantly increasing the image noise level due to shot noise from a stable level of background ambient light.

In some embodiments, the smart phone adapter and retention mechanism may be configured so as to be modularly interchanged with one or more alternative smart phone adapter and retention mechanism(s). The various smart phone adapter and retention mechanisms may be configured such that they have common mounting pins, detents, screws, clasps or other alignment devices as needed. The different smart phone adapter and retention mechanisms may be configured such that the optical center of the respective cameras of the smart phones mounted in appropriate smart phone adapter and retention mechanisms will be properly centered on the optical center of the lateral flow assay fluorescence system, and may be further positioned such that the camera is positioned such that the lens of said camera may appropriately focus the light transmitted to the camera such that the camera of the smart phone may produce an image of sufficient quality for analysis.

Some smart phones have sensors of different sizes, and may have lenses with different focal lengths. A fixed focal system in a lateral flow analysis system may not be capable of providing an image of appropriate size and quality to the range of camera sensors in current use in smart phones. Thus in some embodiments, a collection lens may be associated with some swappable retention mechanism to match focal lengths and or image size between camera and a lateral flow assay fluorescence system. The lateral flow assay fluorescence system may be configured such that more common smart phone cameras need no additional collection lens.

In other embodiments, an adjustable zoom lens may be utilized as a part of the lateral flow assay fluorescence system. The adjustable zoom lens may be configured to accommodate differences in focal lengths and sensor sizes between various smart phones. The zoom lens may be manually adjustable, wherein indicia may be utilized to coordinate adjustment of the adjustable zoom lens and various smart phone cameras; the user may be instructed as to what position to utilize by a printed table, or may instructed by an application associated with the smart phone, wherein the application may interrogate the smart phone to determine the manufacturer and model of smart phone, and instruct the user as to how to adjust the adjustable zoom lens.

After adjustment of the adjustable zoom lens and assembly of the smart phone into the smart phone adapter and retention mechanism, and conjoining of the smart phone adapter and retention mechanism and the lateral flow assay fluorescence system, the smart phone may run a self check to insure that the adjustable zoom lens has been properly adjusted, and inform the user as to the current quality of focus and image size.

In alternative embodiments, an electrically adjustable zoom lens may be utilized, wherein a smart phone application may interactively instruct the lateral flow assay fluorescence system so as to appropriately adjust the electrically adjustable zoom lens. In further embodiments, a camera may have an electrically adjustable focus system, wherein a smart phone application may set the focus, either using a preset value, or by measurement of, for example, fiducials or a control region so as to provide an acceptable focus.

In some embodiments, local heater(s) or Peltier(s) may be utilized to control temperature(s) for one or more regions of the lateral flow device. For example, one temperature may be utilized for a portion of the lateral flow device wherein lysis reagents have been deposited or bound and for a portion of the lateral flow device immediately “upstream” of the lysis reagents. Another temperature may be maintained for a region of a lateral flow device wherein isothermal amplification reagents have been deposited or bound and a region of the lateral flow device immediately “upstream” of the deposited or bound isothermal amplification reagents. A further temperature may be maintained for a detection region.

In a fluorescence system, optics needs to be set up to provide a uniform illumination pattern on the binding regions, while blocking excitation light from the collection optics. Typically this is done using expensive interference filters, and the interference filters are often used in combination with expensive dichroic mirrors. Colored glass filters are less expensive than interference filters, but have less “sharp” filtering characteristics, wherein a “sharp” filter may have a steep slope in the change in transmission or reflection as a function of wavelength. Colored glass filters often have some autofluorescence, wherein when a filter may be utilized to filter, for example, an excitation light source, the excitation light may generate fluorescence within the filter. Plastic filters typically are very inexpensive, but they typically have even more gradual filtering characteristics than colored glass filters.

In some embodiments, plastic filters may be utilized in front of a colored glass filter. While the plastic filter sharpness is worse, the plastic filter may attenuate the excitation light sufficiently to minimize the amount of autofluorescent light generated in the colored glass filter to a level acceptable in for a lateral flow assay. In alterative embodiments, a plastic filter may be utilized after a glass filter to remove autofluorescence produced by a glass filter used, for example, for an excitation filter.

Smart phones are commonly available; the built in camera in a smart phone may be utilized to capture fluorescence images. In some embodiments, a smart phone may be integrated with an optics module that provides illumination of a lateral flow strip, and provides filters and optics to collect fluorescence from fluorescent reporters used in the lateral flow assay. In some embodiments, an illumination device may be designed to work with an adapter. The adapter may be fabricated with features that appropriately position a smart phone relative to the illumination device, allowing the illumination device to work with different smart phones. In other embodiments, a lateral flow reader may be configured to hold and secure a smart phone directly to said lateral flow reader without an adapter module.

In some embodiments, an illumination device may provide off axis illumination of the lateral flow binding region(s). In some embodiments, illumination light may be provided by a LED. The LED light output may be controlled via a smart phone or by separate hardware. The LED light may be focused and/or diffused to produce a reasonably uniform concentrated beam over the binding region(s). This focusing and/or diffusing may be accomplished using standard optical lenses, Fresnel lenses, mirrors or other optical components. The illumination device may have light blocking features to prevent ambient light from interfering with the measurement.

In other embodiments, the illumination device may provide back side (transmission) illumination of the lateral flow binding region(s). Back side illumination (transmission) becomes more practical when a lateral flow substrate or membrane is kept wet with a fluid, and the index of refraction of the fluid substantially matches the index of refraction of the lateral flow substrate or membrane.

### Software for Normalization and Camera Control

In some embodiments flat field compensation may be effectuated to compensate for variations in excitation uniformity, collection efficiency vs. position in image, pixel gain, pixel response both in QE and angular response at desired wavelengths, debris in the optical path, and any other variations in the response of the optical system which may vary by position.

In some embodiments, the software, which may be a software application which may run on a smart phone utilized to capture images associated with an assay may perform a detection sensitivity check using a calibration area on a lateral membrane or may perform such a test on a separate test target.

In further embodiments, the software may perform a check of the detection resolution, using for example, fiducials which may be printed on a lateral flow membrane. In further embodiments, software may be utilized to with fiducials on a membrane or test target to determine whether the magnification of the image is appropriate for a particular assay. In additional embodiments, the software may utilize a membrane or test target to check and map debris in optical path, wherein the locations of pixels which are obscured or degraded may be stored, and data associated with those locations may be disregarded in a later analysis, or if the number and position of obscured or degraded pixels may prevent a desired assay from giving a result with a desired confidence value, the user may be warned so as to prevent inappropriate use of a camera/system which is incapable of performing as desired.

In some embodiments, software may utilize fiducials to determine whether a camera and system combination generates excessive optical distortion, such as pincushion distortion. In further embodiments, fiducials may be utilized to check the position and orientation of a membrane or substrate, and to warn the user of any inappropriate alignment.

In some embodiments, software which may be associated with the camera may check and or set the shutter and ISO control capabilities of camera, and of the camera within the system, so as to insure proper capabilities of a combined system and camera for a particular assay.

In some embodiments, software which may be associated with the camera may check and or set the output power level of a system excitation LED and or the transmission of the system optics using a test strip of controlled fluorescence; in further embodiments, the software may check for excessive background signal levels, and may additionally capture background levels for later subtraction from assay images. In some embodiments, the software may check background levels with any excitation LEDs off, particularly with long exposure times, so as to determine dark current and camera offset levels, and by so doing, may check for dark current and hot pixels; the location of any hot pixels may be mapped and stored; in subsequent analysis the software may determine whether the hot pixels may have a detrimental effect that may reduce the confidence level associated with an assay, and may warn the user as to the reduction in the confidence level, including instructing the user to disregard resulting data as a result of the determination of the confidence level which may be degraded from hot pixels, or may be degraded from a variety of other factors as determined by the software. In some embodiments, the software may check background levels with any excitation LEDs off, particularly with short exposure times so as to determine the read noise of camera, and may determine the read noise for each pixel or output tap for CMOS and CCD devices respectively.

In some embodiments, the software may check background levels with any desired excitation LEDs on, particularly with long exposure times, so as to determine any background light leaks or autofluorescence which may exist in the system.

In some embodiments, a target may utilize the back side of a membrane or another material provided with membrane; in other embodiments a target may utilize a cover for the membrane.

In some embodiments, software may be utilized with a target material which may have uniform in band fluorescence wherein the system may illuminate and capture image(s) and may thence normalize the images to a maximum of one, and may then divide on a pixel by pixel basis to remove illumination and collection non-uniformity, thus flat fielding an image. In other embodiments, other specific normalization methodologies may be utilized to provide optical normalization of images so as to provide improved quantification.

In some embodiments, software may be utilized with a target material which may have uniform reflectivity or out of band fluorescence, or an additional LED, which may be selected so as to pass through the emission filter(s), may be utilized, wherein image(s) may be acquired without one of the filters such as the emission filter, or sufficient transmission of the supplementary LED may serve to provide sufficient light through the excitation filter set, so as to perform desired checks. Any supplementary LED may need to create an illumination profile substantially similar to that of the main excitation LED, or alternatively, a calibration between the supplementary LED illumination profile and the main excitation LED may be utilized to perform desired checks and or calibrations.

In some embodiments, software may be utilized with a target with in band fluorescent spots which may be scattered over the surface of the target so as to cover a sufficient area so as to capture any system non-uniformity.

In some embodiments, checks of the performance of a camera in the system may be utilized to determine whether a particular camera is suitable for use with a particular assay, particularly wherein some assays may have more stringent requirements for resolution sensitivity, image size, or image quality.

In some embodiments, software may be utilized with a fiducials interspersed with binding regions so as to permit accurate determination of the locations of the binding regions, allowing greater accuracy and sensitivity of the assays.

In some embodiments, software may be utilized with a variable power LED, which may be an LED in addition to the excitation LED, so as to set and lock the shutter speeds and ISO settings of the camera, which may be inaccessible directly to the software, but may be accessible as a result of changing the light which the camera may sense until a desired shutter speed and setting may be obtained. In further embodiments, the variable power LED or the excitation LED may be utilized in combination with one or more calibration standards to set shorter shutter speeds, wherein a localized area within the image which may correspond to the location of one or more calibration standards may be utilized to set the shutter speed.

In some embodiments automated reporting using the phone or data connections from a smart phone may be used to collect information for disease tracking, QC of testing, etc.

In some embodiments, software may be utilized with a camera in the smart phone to enter test information such as lot number, expiry date, etc. which may utilize one or two dimensional barcodes; the smart phone may further be utilized as a data entry mechanism in order to associate a patient, doctor, location or other parameters with a set of data.

In some embodiments, software may be utilized with the smart phone to capture GPS location and may associate the GPS location with any assay results.

### EXAMPLES

### Example 1

### Decomplexation

A lateral flow assay illustrating the use of a decomplexation region was performed on commercially available hCG lateral flow strips purchased from Formosa Medical®. The test was called the Wondfo 50 (HCG) Pregnancy Test Strip; the distributor was Amazon. Goat polyclonal anti-hCG and â-hCG were purchased from Scripps Laboratories (San Diego, Calif.). Glass fiber was manufactured by Millipore Corporation (Bedford, Mass.). Backing material was obtained as a sample from DCN Diagnostics (Carlsbad, Calif.).

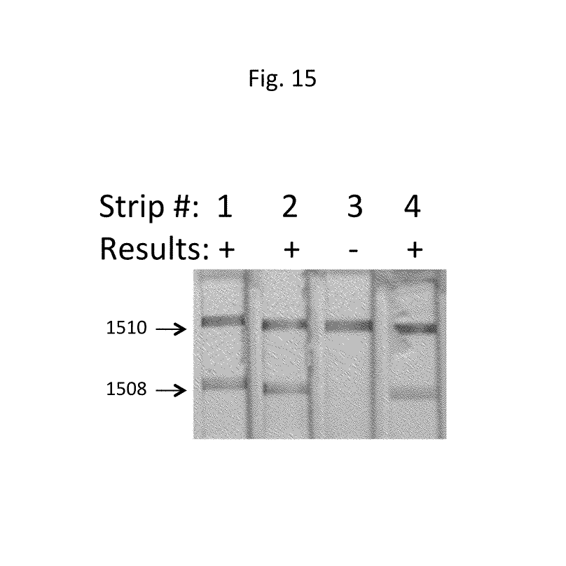

Extra lengths of backing and glass fiber (3 mm×6 cm) were appended to the strips. To create the decomplexation region, citric acid solution (3 uL, 1 M) and Tris base solution (5 uL, 3 M) were applied to the extensions 3 and 8 mm from the sample end and dried down. Sample (5 uL, 0.13 ug/mL hCG or 5 uL of a mixture of 0.13 ug/mL hCG and 5 mg/mL goat anti-hCG) was applied to the strips directly on the decomplexation region followed by immersing the end of the strip in eluent (80 uL of 1% bovine serum albumen in phosphate buffered saline). The results are shown in FIG. 15. Strip 1 shows both control regions 2210 and test regions 2208 when uncomplexed analyte is used. The presence of a decomplexation region does not affect the intensity of the stripes (strip 2). The presence of antibody to complex the analyte gives a negative test result as shown in strip 3. The presence of a decomplexation region and complexed analyte gives a positive test result as shown in strip 4.

FIG. 15 shows the results from test strips with appended backing and glass fiber. Strip 1: free analyte, without decomplexation region; strip 2: free analyte, with decomplexation region; strip 3: complexed analyte, without decomplexation region; strip 4: complexed analyte, with decomplexation region.

### Example 2

### Quantitative Fluorescent Detection

### Materials

Biotinylated BSA and streptavidin were purchased from Thermo Fisher Scientific (Rockford, Ill.). R-PE streptavidin and Alexa Fluor streptavidin were purchased from Life Technologies (Carlsbad, Calif.). BSA was purchased from Sigma-Aldrich (St. Louis, Mo.). Brilliant Violet 605 streptavidin was purchased from BioLegend® (San Diego, Calif.). Chromeo 494 streptavidin was purchased from Active Motif® (Carlsbad, Calif.). Atto™ 465 streptavidin and Atto™ 430-LS streptavidin were purchased from Atto-tec (Siegen, Germany). Gold-labeled streptavidin was purchased from Innova Biosciences (Cambridge, UK). Biotin-X-NHS ester was purchased from AAT Bioquest® (Sunnyvale, Calif.). Goat polyclonal anti-hCG, beta hCG, and mouse monoclonal anti-hCG were purchased from Scripps Laboratories (San Diego, Calif.). Lateral flow materials were samples from Millipore Corporation (Bedford, Mass.) and GE Healthcare (Buckinghamshire, UK).

Colored glass optical filters were purchased from Thor Labs (Newton, N.J.). Interference filters were purchased from Chroma Technologies Corp® (Bellows Falls, Vt.). Plastic filters were purchased as a booklet from Edmund Optics (Barrington, N.J.). The LEDs (Phillips Luxeon® Star) and LED optics (except 405 nm LED) were purchased from Quadica Developments Inc (Brantford, Ontario). The 405 nm LED and reflector was purchased from SuperBrightLEDs.Com® (Saint Louis, Mo.). An iPhone® 4 was purchased from Apple® (Cupertino, Calif.). ProCamera was purchased from Cocologics (Mannheim, Germany) through the Apple® App store. ImageJ software was downloaded from the NIH website (National Institutes of Health, Bethesda, Md., USA, http://imagej.nih.gov/ij/).

### Optics Breadboard Design and Construction

Except were noted the following description of the breadboard is specific for analysis of R-phycoerythrin (R-PE).

FIG. 14 schematically illustrates the optical breadboard, wherein light emitted from an excitation LED and associated reflector 1476 passes through an aperture and excitation filter 1486 and is focused by an excitation lens 1492 before illuminating the substrate or membrane associated with a support which may be a glass slide 1446. Fluorescent light emitted from bound labeled target complexes or other fluorescent sources is collected by a collection lens 1482 and passes through an emission filter 1484 before being imaged into a cell phone and associated camera 1478.

To facilitate easy setup modification and allow use of 1″ optics and filters the optics breadboard (BB) was constructed using 30 mm cage components (Thor Labs, Newton, N.J.). The cage components were secured to an aluminum plate positioning optics as shown in FIG. 14 allowing motion of one plate to clamp the smart phone. The excitation source was a 505 nm LED providing 122 lm at 700 mA (SR-01-E0070, Quandrica Developments, Brantford, Ontario). The LED current was controlled by a 700 mA externally dimmable DC driver (A011-D-V-700, LEDdynamics™ Quadrica Developments) powered by eight AA batteries with holder (Mouser Electronics®). A 20 k Ohm potentiometer (652-3386P-1-203LF, Mouser Electronics®) was used to control the LED current (normally set to full (700 mA) except when setting exposure). A power switch (611-CA22J72207PQ, Mouser Electronics®) was provided to prevent draining of the batteries when not in use. The LED was mounted to the cage support endplate using precut thermal adhesive tape (LXT-S-12, Quandrica Developments) with a 7°, 11 mm reflector (Dialight™). The excitation filter was provided by two 0.003″ thick plastic films (Supergel #69 brilliant blue, Rosco). The excitation beam was focused using a 25 mm diameter, 25 mm FL acrylic lens (NT48-170, Edmund Optics). A schematic of the optics breadboard is shown in FIG. 14.

The scattered emission light was first filtered using a single 0.003″ thick plastic film (Supergel #15 Deep Straw, Rosco) along with an 2 mm thick, Schott OG570 colored glass filter (FGL570, Thor labs). The emission light was semi-collimated using a 25 mm diameter, 25 mm FL acrylic lens (NT48-170, Edmund Optics) for collection using the smart phone (iPhone® 4). Apertures were hand cut out of black plastic and the system was shielded from room light using a hand fabricated black foam core box.

Image capture was performed using the ProCamera app with the following settings: Lightbox off, expert mode on, self timer 5 sec. The exposure time (varied) and ISO settings (always set to ISO 80) were set by trial and error on a selection of points on the image from a fluorescent target (paper marked with orange highlighter) and locked. Once the camera settings were locked the potentiometer was set for max current (700 mA) and images were taken of nitrocellulose mounted to standard 1″×3″ glass slides that were temporarily secured using double sticky tape to the cage endplate.

Several variations of the optics breadboard were used to analyze different fluorescent dyes. Several different LEDs were utilized, and different emission and excitation filter configurations were utilized to optimize the system for each dye. Four different LEDs were used, with center wavelengths ranging from 405 to 530 nm, although any center wavelength LED could have been utilized, including, UV LEDs, red LEDs, near infrared LEDs, or infrared LEDs. Similarly, various filters with different filter types, centers, thicknesses were utilized for both excitation and emission, including expensive interference filters, colored glass filters and plastic filters.

### Image Analysis

Captured images were analyzed using ImageJ software. Images were cropped and rotated so the flow direction was horizontal. The images were converted to RGB format and the appropriate color selected (red for R-PE, green for colloidal gold). A freehand line was drawn around the fluorescent zone and the intensity, area, min and max were collected using the measure icon. A rectangle was drawn and used for a plot profile across the illuminated area. Column averages to the left and right of the spot were used to find a baseline for the data. If different exposure times were used the signals were appropriately scaled. The total signal over baseline was calculated and then plotted on log-log scales with a power fit trend line using Excel. For colloidal gold the same method was used, except the total signal below the baseline (absorbance) was used.

### Nonspecific Binding Measurement

Dye-labeled streptavidin was diluted to create a two-fold dilution series in 1× phosphate buffered saline (PBS) in the range of 0.63-40 μg/mL. To generate the spots for the signal data, the dilution series was spotted (1 μL) on untreated nitrocellulose dried and mounted onto glass slides. To generate strips for the nonspecific binding data, strips of nitrocellulose (5 mm×20 mm) were initially immersed into 5% bovine serum albumin in PBS for 30 min, rinsed and dried. The strips were then immersed into 0.5 mL of the dilution series for 20 min, rinsed in 1×PBS, dried and mounted on glass slides.

Lateral Flow with Streptavidin, Biotinylated BSA, R-PE-Streptavidin and Streptavidin, Biotinylated BSA, Gold-Streptavidin

Nitrocellulose (Millipore HiFlow Plus HFB13502) was cut (4 cm×4 cm) and mounted onto an adhesive cardboard backing 6 mm from the edge. Glass fiber conjugate pad (Millipore GFCP20300) was cut into a rectangle (8 mm×4 cm) and mounted on the edge of the backing, overlapping the nitrocellulose by 2 mm. Absorbent material (GE Healthcare, CF3) was cut (4×4 cm) and mounted on the backing, overlapping the nitrocellulose by 2 mm. The assembly was cut into 4 mm wide strips. Streptavidin was spotted at 4 mg/mL in 0.5 μL aliquots 1 cm above the absorbent pad. A four-fold dilution series of biotinylated BSA in 1% BSA/PBS was prepared, in concentrations ranging from 63 pg/mL to 16 μg/mL. The strips were dipped successively into 20 μL of each concentration of the dilution series, 20 μL of R-PE streptavidin (0.01 mg/mL in 1% BSA/PBS), and 50 uL 1% BSA/PBS. The strips were air-dried and mounted on glass slides.

Lateral Flow with Anti-hCG, Beta-hCG, Biotin-Anti-hCG/R-PE-Sav and Anti-hCG, Beta-hCG, Biotin-Anti-hCG/Gold-Sav

Strips were constructed as described above. Mouse monoclonal anti-hCG was biotinylated with biotin-X-NHS at pH 9.2 and excess reagent removed on a Sephadex™ G-25 column. Goat polyclonal anti-hCG was spotted at 4 mg/mL in 0.5 μL aliquots 1 cm above the absorbent pad. A four-fold dilution series of hCG in 1% BSA/PBS was prepared, in concentrations ranging from 1000 ng/mL to 63 pg/mL. The strips were dipped successively into 20 μL of each concentration of the dilution series, 20 μL of a mixture of 0.01 mg/mL R-PE streptavidin and 0.005 mg/mL biotinylated mouse monoclonal anti-hCG in 1% BSA/PBS, and 50 μL of 1% BSA in PBS. The strips were air-dried and mounted on glass slides.

### Results

### Survey of Fluorescent Reporters; Ratio of Signal to Nonspecific Binding.

Many fluorescent entities are available commercially, and conveniently, many are available as streptavidin conjugates. Fluorescent compounds can be divided into two types, soluble “small molecules” and particles, such as fluorescent latex beads, quantum dots or europium chelates. These experiments were focused on understanding the soluble type of fluorescence molecules. Initial experimentation utilized dot blots with spotted down biotinylated BSA and detection of bound dye-labeled streptavidin, but high levels of background fluorescence was limiting sensitivity for several of the dyes, requiring a quantitative approach to characterize the nonspecific binding of each of the dyes to blocked nitrocellulose.

Quantification to allow comparison of the nonspecific binding characteristics of various dyes relied on determining the ratio of the signal to the nonspecific binding (NSB) signal for each dye conjugated to streptavidin. The signal from spotting a fixed volume (1 μL) of a dilution series of a dye-labeled streptavidin and the signal from dipping pre-blocked nitrocellulose in the same dilution series were plotted. Linear fits to the data were calculated using Excel and the ratio of the two slopes gave a unitless number, the ratio of signal to NSB. This number is independent of the sensitivity of detection of each system. This system independence is necessary since the various dyes require different LEDs and filters.

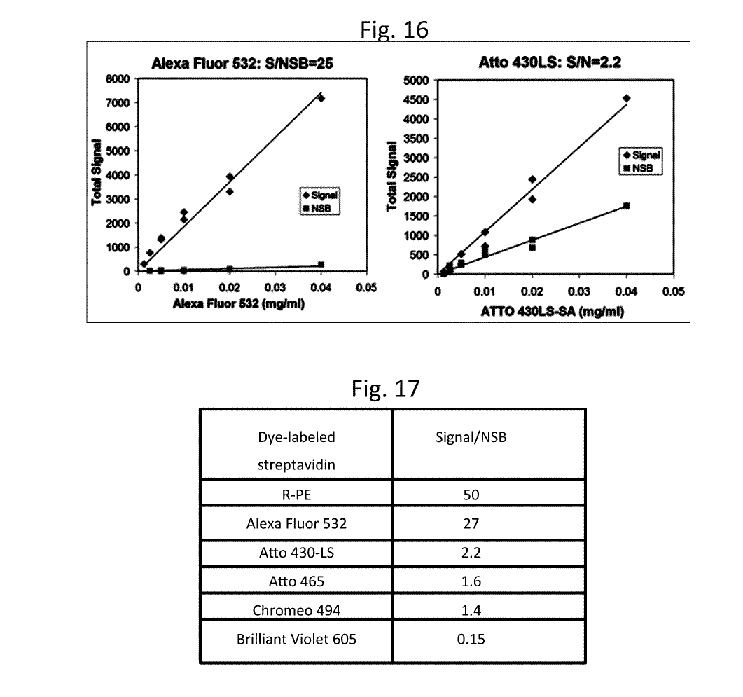

Shown in FIG. 16 are the graphs of signal and NSB data for two of the dyes, Alexa Fluor 532 and Atto 430LS. Alexa Fluor 532 has a good ratio of signal to nonspecific binding (S/NSB) compared to Atto 430LS. Each fluorophore is conjugated to streptavidin, spotted on nitrocellulose and the signal read in the breadboard (signal, blue diamonds). Strips of nitrocellulose that have been blocked with BSA were immersed in each solution and read in the breadboard (nonspecific binding, magenta squares). The ratios of the two slopes are reported as the S/NSB ratio.

FIG. 17 shows the ratio of signal to nonspecific binding for fluorescent dyes conjugated to streptavidin, for all the dyes analyzed, in table form. Surprisingly, even though all the dyes were very water-soluble, they showed a wide range in ratios of signal to NSB. Brilliant Violet 605™ streptavidin was extraordinarily “sticky”, actually producing greater signal in the nonspecific binding mode than the signal mode for each dilution of dye-labeled streptavidin. Alexa Fluor 532 streptavidin and R-PE streptavidin were the least sticky. It is clear from these results that besides the inherent brightness of a fluorescent dye, the ratio of signal to NSB is a key characteristic in determining the utility of a dye in lateral flow. A dye with a high ratio of signal to NSB will have a good dynamic range since high concentrations of dye can be used to saturate high concentrations of analyte without causing too much background for low concentrations of analyte.

### Fluorescence Reader System

A fluorescence lateral flow system of strip and reader that is both low-cost and high-performance is desired that would be an accessory to a smart phone. See, for example U.S. Pat. Nos. 8,011,228 and 7,371,582 which are incorporated by reference herein in their entirety for all purposes. To achieve this, a fluorescent reporter that had a long Stokes shift is desired; that is, where the excitation maximum is well separated from the emission maximum. If the Stokes shift is greater than approximately 70 nm, extremely low cost colored plastic or colored glass can replace costly interference filters that are typically used in fluorescence readers. For the light source, LEDs were used in a variety of wavelengths. Instead of a scanning system to detect the signal, we used the camera in a common smart phone, the iPhone® 4. This allowed variation in the length of exposure, extending the dynamic range of the assay. Ultimately, data analysis may be done on the mobile device utilizing a mobile image analysis application. For data shown here, the images were downloaded to a computer and used ImageJ for the analysis.

Some examples of long stokes shift dyes which may be useful for LFAs include phycoerythrin, phycoerythrin-Cy 7, phycoerythrin-Cy 5.5, phycoerythrin-Texas Red, propidium iodide, PerCP (peridinin chlorophyll protein), PerCP-Cy5.5, FITC (Fluorescein isothiocyanate), allophycocyanin, allophycocyanin-Cy 7, Alexa Fluor 430, and DAPI (4′,6-diamidino-2-phenylindole). As described hereinafter in the experimental section, such dyes may be utilized to provide dynamic ranges of greater than three orders of magnitude. Such dyes may have a ratio of signal to nonspecific binding of at least 5, of from 5 to 10, of from 10 to 15, of from 15 to 20, or greater than 20.

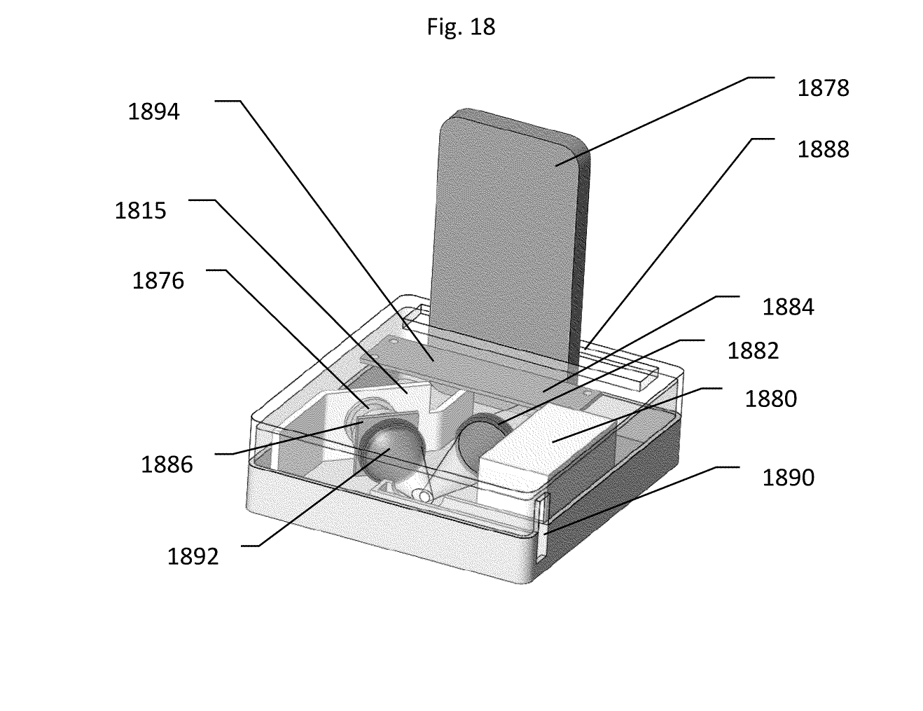

Functions for such a system include LFIA detection, analysis and communications. Shown in FIG. 18 is the design for such a reader; for clarity internal baffles are not depicted. The unit is activated by a power switch on a PCB 1894 which controls the LED current and on time. Power is supplied by a battery pack 1880. The LED and associated reflector 1876 are positioned by the LED heat sink 1815. Light from the LED passes through the excitation filter 1886 prior to being focused by the optional excitation lens 1892. Light is collected by the collection lens 1882 and is thereby focused through the emission filter 1884 (not clearly visible under PCB 1894) and into the camera of a cell phone and associated camera 1878. The phone is held in position by a phone adapter 1888, which allows for the use of different types of smart phones, or may be held directly, wherein different top pieces which may include emission lens(es) may comprise an integrated molded top piece. A slot 1890 is provided for the insertion and removal the lateral flow assay assembly. The slot may comprise baffles or flexible material useful to prevent ambient light from entering into the lateral flow reader and compromising image data. A lateral test strip holder may be configured to interlock with features of said slot so as to better effectuate ambient light blocking.

Lateral Flow with a Sandwich of Streptavidin, Biotinylated BSA, and Labeled Streptavidin

Following the method of Juntunen et al. Anal. Biochem. 2012, 428, 31-38, streptavidin conjugates were tested using a simplified lateral flow format. The pad containing the labeled reagent was omitted; instead, a simplified lateral flow strip consisting of feeding pad, nitrocellulose and absorption pad on a cardboard backing was constructed. A spot rather than a stripe of reagent was applied to the nitrocellulose. The strip was dipped into three successive solutions of analyte, labeled reagent, and then buffer. Each of these solution contained 1% BSA to prevent nonspecific adhesion of the proteins to the nitrocellulose. The strips were then allowed to dry and read on the breadboard. This format was used to compare fluorescent (R-PE) and absorbance (gold) assays in which all components were identical, except the labeled streptavidin.

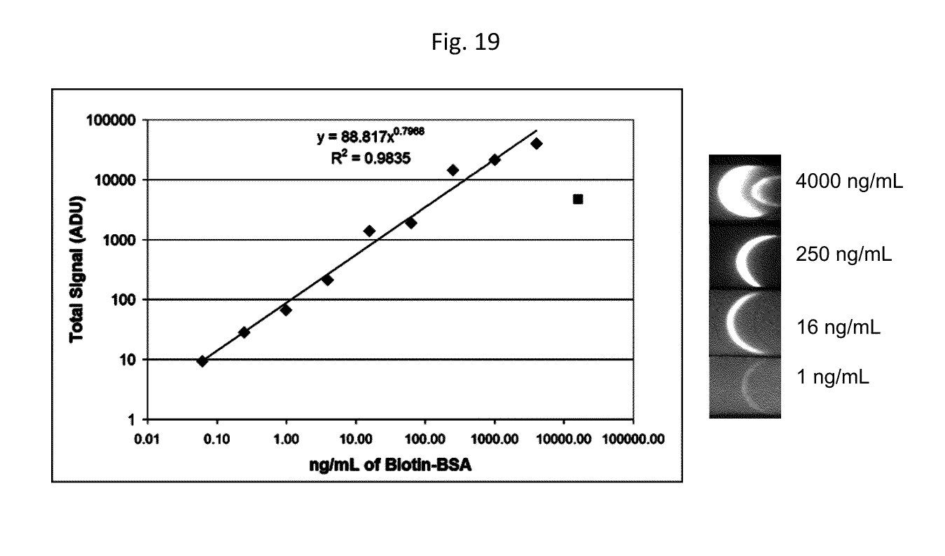

FIG. 19 shows fluorescence lateral flow images and plots resulting from a fluorescence lateral flow assay that utilized a sandwich system of streptavidin, biotinylated BSA, and R-PE-labeled streptavidin. Spots rather than the conventional stripes of streptavidin were applied to the nitrocellulose and allowed to dry, resulting in the round or crescent shapes. The moon shapes result from antigen binding to the first bound antigen the antigen interacts with; thus as the flow interacts with a round spot of bound antibodies, a crescent shape is formed. A four-fold dilution series of biotinylated BSA in 1% BSA was prepared. Each strip was dipped successively into 20 μL of the dilution series, then into 20 μL of R-PE streptavidin, then into 50 μL 1% BSA. The results show a very wide dynamic range (0.1-4000 ng/mL) and sensitive detection. At the upper end of the concentration range (16,000 ng/mL), the signal is no longer linear. The loss of linearity is due to the “prozone effect” that occurs when the concentration of analyte is high enough to saturate both antibodies, precluding the formation of the antibody-analyte-antibody sandwich. Images were obtained in the previously described breadboard equipped with an iPhone® 4 and ProCamera app. Image analysis was done with Image J and the results plotted.

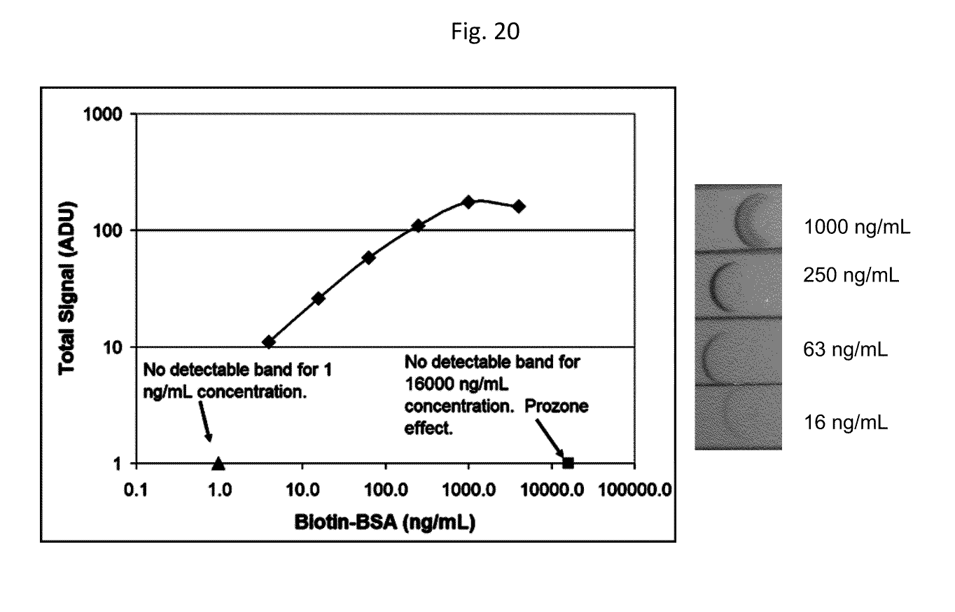

FIG. 20 shows the analogous absorbance lateral flow assay images and plots resulting from the substitution of colloidal gold for the R-PE on streptavidin and flash photography instead of fluorescence detection. In a similar fashion to the fluorescence assay, the strips were spotted with streptavidin, followed by dilutions of biotinylated BSA, followed by gold-labeled streptavidin, followed by buffer were absorbed on the strips. Images were obtained with the camera of an iPhone® 4. Image analysis was done with Image J and the results plotted. The absorbance system has a narrower useful concentration range as well as a less sensitive limit of detection. Compared to the fluorescence data, the absorbance data has a smaller useful dynamic range of 4-1000 ng/mL of biotinylated BSA. The dynamic range of the signal is also smaller; the difference between the highest and the lowest signal is only 10-fold. The prozone effect is observed at 16,000 ng/mL as a complete absence of signal.

Lateral Flow with a Sandwich of Polyclonal Anti-hCG, hCG, and Biotinylated Monoclonal Anti-hCG/R-PE Streptavidin

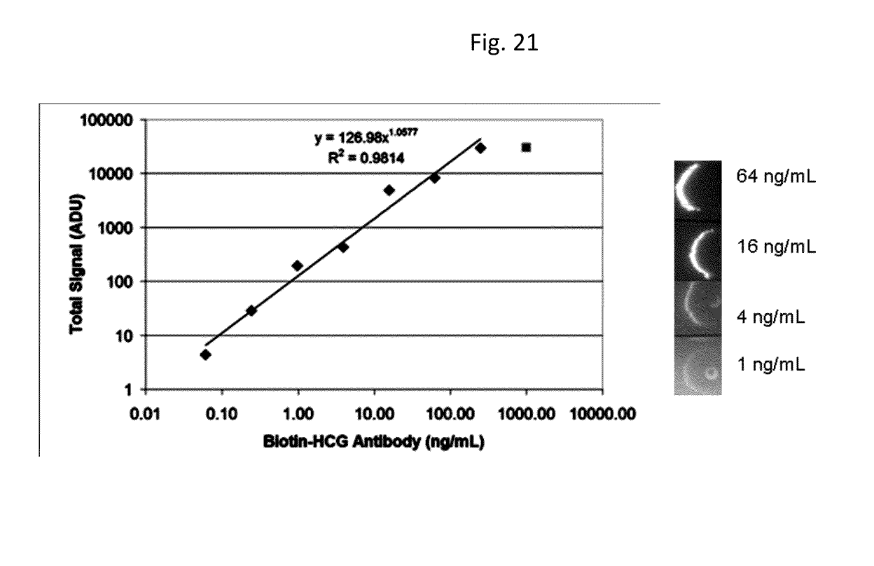

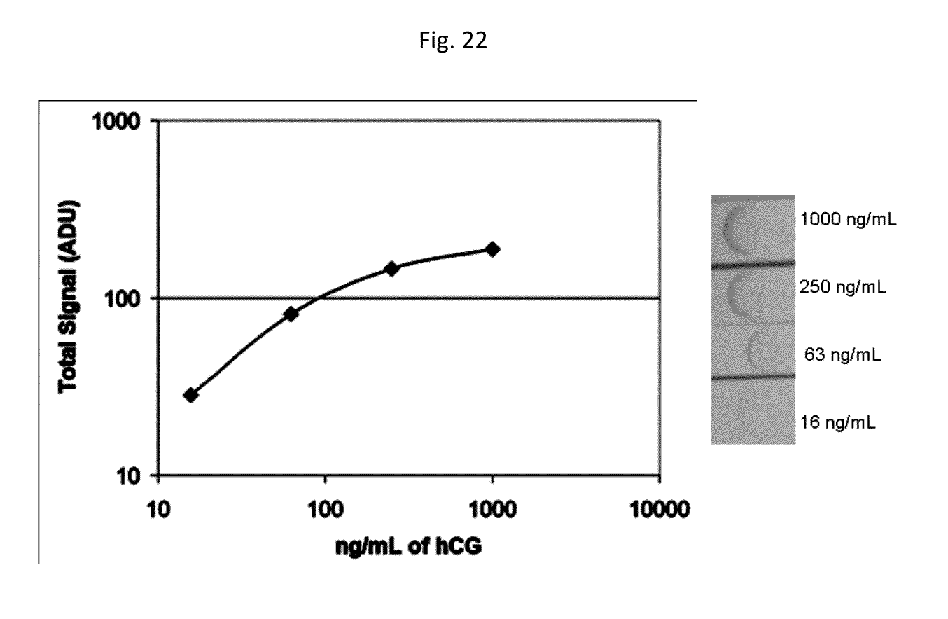

Analysis of human chorionic gonadotropin (hCG) was also performed with the simplified lateral flow system with both fluorescence and absorbance measurement. The sandwich system for fluorescence consisted of polyclonal goat anti-hCG spotted on the strip, anti-hCG as the analyte, and biotinylated mouse monoclonal anti-hCG mixed with R-PE streptavidin. The results of testing strips in a four-fold dilution series for fluorescence lateral flow analysis of hCG are shown in FIG. 21; while results for a gold absorbance lateral flow analysis is shown in FIG. 22. The prozone effect is evident at 1000 ng/mL with a non-linear data point. Evidence of the pipette tip used for spotting the goat antibody appears as a fluorescent spot, perhaps due to a high local concentration of antibody as a result of the pipette tip touching and indenting the lateral flow membrane.

### Photobleaching of Alexa Fluor 532 and of R-PE

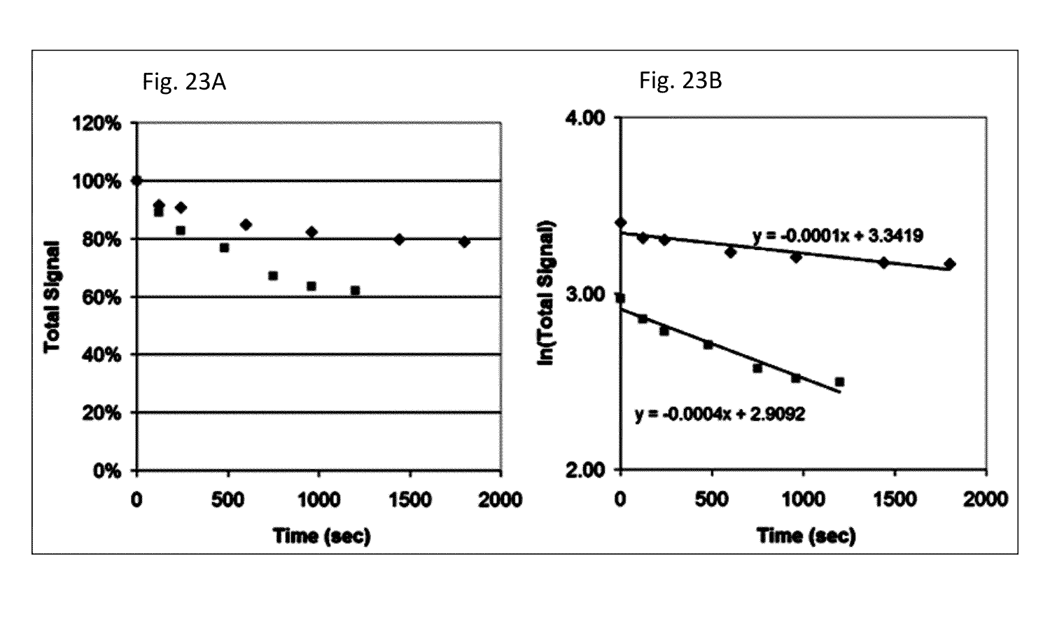

R-PE is reported to be less photostable than organic dyes. The photostability was tested in our breadboard by illuminating spots of R-PE streptavidin and spots of Alexa Fluor streptavidin and recording the loss of signal over time. FIG. 23 shows plots of signal vs. time for both dyes. Under constant LED illumination, Alexa Fluor 532 is more stable than R-PE. Both are expected to be sufficiently stable under normal storage conditions of lateral flow strips.

FIG. 23A FIG. 23B graphically depict photobleaching levels of R-PE streptavidin and Alexa Fluor 532 streptavidin. The compounds were spotted on nitrocellulose and exposed to constant illuminations with a 505 nm LED. Images were collected at time intervals corresponding to illustrated data. FIG. 23A graphically depicts data that was normalized to the initial values for both R-PE streptavidin and Alexa Fluor 532 streptavidin. FIG. 23B graphically depicts a plot of the natural logarithm of the signal provided a t_{1/2} of 2,000 sec for R-PE and 7,000 sec for Alexa Fluor 532.

We have evaluated several soluble fluorescent dyes conjugated to streptavidin and determined a method to evaluate the signal vs. nonspecific binding characteristics of these compounds on lateral flow materials. The method is simple and should be readily applicable to fluorescent particles, such as latex beads and quantum dots, as these are also readily available conjugated to streptavidin. We have built an illumination device coupled with a smart phone to allow detection and analysis of R-PE in lateral flow format for various fluorescent dyes. We show superior performance compared to colloidal gold in lateral flow analyses of biotinylated BSA and hCG in both sensitivity and dynamic ranges of analyte concentration and signal level. Further improvement can likely be achieved by deposition of antibody on the nitrocellulose in stripes rather than by manually spotting, direct attachment of R-PE to the antibody rather than via streptavidin/biotin, and further reduction in nonspecific binding by evaluation of different materials, buffers and blocking agents. A custom smart phone application can allow longer exposure times (currently limited to 1/15 sec by the ProCamera application). Additional improvements are likely using image processing techniques such as flat field correction and combining of multiple images. Testing of a lateral flow strip complete with a conjugate pad to hold and deliver fluorescent antibody will be necessary to determine reagent stability and performance.

There are both advantages and disadvantages to using fluorescence in lateral flow. The advantages include higher sensitivity, and wider dynamic ranges in analyte concentration and in signal level. The disadvantages include the requirement of a reader since the fluorescent signals are only visible to the eye at a high concentration. In addition, the chemistry of conjugation of fluorescent materials requires single or multistep covalent conjugation chemistry. Attachment of antibodies to colloidal gold, by contrast, is often achieved by pH dependent passive absorption.

There are also advantages and disadvantages to the use of a smart phone as the detector and analyzer. The advantages include readily upgradable applications, the ability to instantly store data in the cloud, facilitating the automation of disease tracking, and the compact size and ubiquity of smart phones, obviating the need for a bulky computer. Disadvantages include the inherent difficulty in obtaining FDA/CE approval due to the constantly changing standards. These changing standards also result in the requirement for a very flexible interface in the illumination device.

In summary, although existing gold lateral flow strips are robust, simple and good for positive or negative determination, use of fluorescence offers the advantages of quantitation and increased sensitivity when these requirements are needed. Examples of where these requirements may offer significant advantage include quantitation of IgG and IgM to distinguish between primary and secondary dengue infections; detection of low parasite levels prior to malaria recrudescence; accurate quantitation of cardiac markers; and quantification and identification of environmental contaminants. We believe we have shown a method to offer these advantages in a point-of-use setting.

It will be apparent to those skilled in the art that various modifications and variations can be made without departing from the spirit or scope of the invention. Thus, it is intended that the disclosed embodiments cover modifications and variations that come within the scope of the claims that eventually issue in a patent(s) originating from this application and their equivalents. In particular, it is explicitly contemplated that any part or whole of any two or more of the embodiments and their modifications described above can be combined in whole or in part.

What is claimed is:

1. A lateral flow test strip for detecting analyte levels in a sample comprising:
a sample application region;
a decomplexation region for dissociating analyte-antibody complexes in the sample;
a conjugate region comprising a detection antibody that selectively associates with the analyte;
a flow region; and
a test line comprising immobilized test antibody.

2. The lateral flow test strip of claim 1, further comprising a neutralization region comprising neutralizing agents that neutralize the decomplexation reagent.

3. The lateral flow test strip of claim 1 or 2 further comprising an elution reagent application region on the strip upstream of the sample application region.

4. The lateral flow test strip of claim 1 or 2 wherein the strip is configured such that the elution reagent combined with the sample is added to the sample application region of the strip.

5. The lateral flow test strip of claim 1 or 2 wherein the decomplexation region comprises an acidification reagent that lowers the pH of the sample as the sample passes through the decomplexation region.

6. The lateral flow test strip of claim 2 wherein the decomplexation region comprises an acidification reagent that lowers the pH of the sample as the sample passes through the decomplexation region, and wherein the neutralizing reagent comprises a base that raises the pH of the sample as it passes through the neutralization region.

7. The lateral flow test strip of claim 5 wherein the acidification reagent brings the pH of the sample to less than about 5.

8. The lateral flow test strip of claim 5 wherein the acidification reagent brings the pH of the sample to less than about 4.

9. The lateral flow test strip of claim 5 wherein the acidification reagent brings the pH of the sample to less than about 3.

10. The lateral flow test strip of claim 5 wherein the acidification reagent comprises citric acid, glycine-HCl, or tartaric acid.

11. The lateral flow test strip of claim 5 wherein the acidification reagent comprises a polymeric cation exchanger in the acid form.

12. The lateral flow test strip of claim 5 wherein the acidification reagent comprises a carboxylic acid, a sulfonic acid, a phosphoric acid or a phosphonic acid.

13. The lateral flow test strip of claim 1 or 2 wherein the decomplexation region comprises a detergent.

14. The lateral flow test strip of claim 13 wherein the detergent comprises sodium dodecyl sulfonate.

15. The lateral flow test strip of claim 1 or 2 wherein the decomplexation region raises the salt concentration in the sample for decomplexation.

16. The lateral flow test strip of claim 15 wherein the salt comprises lithium chloride, magnesium chloride, or sodium thiocyanate.

17. The lateral flow test strip of claim 1 or 2 wherein the decomplexation region provides an organic solvent into the sample for decomplexation.

18. The lateral flow test strip of claim 17 wherein the organic solvent comprises ethylene glycol.

19. The lateral flow test strip of claim 1 or 2 wherein the decomplexation region comprises a chaotropic agent.

20. The lateral flow test strip of claim 19 wherein the chaotropic agent comprises urea or guanidine-HCl.

21. The lateral flow test strip of claim 1 wherein the decomplexation region is a region that is heated.

22. The lateral flow test strip of claim 21 wherein the heating is provided by a compound that gives off heat when it comes in contact with the elution reagent.

23. The lateral flow test strip of claim 21 wherein the heating is provided by an electric heater.

24. The lateral flow test strip of any of the claims above wherein the detection antibody comprises a fluorescent label.

25. The lateral flow test strip of any of the claims above wherein the decomplexation region and the sample application region are coextensive.

26. The lateral flow test strip of any of the claims above wherein the neutralization region is coextensive with the conjugate region.

27. A lateral flow test strip for detecting analyte levels in a sample comprising:
a sample application region;
a decomplexation region comprising a dissociating reagent for dissociating analyte-antibody complexes in the sample,
a conjugate region comprising a detection antibody that selectively associates with the analyte; wherein the sample is mixed with an elution reagent that comprises components which result in the neutralization of the dissociating reagent before it reaches the conjugate region;
a flow region; and
a test line comprising immobilized test antibody.

28. The lateral flow test strip of claim 27 further comprising an elution reagent application region on the strip upstream of the sample application region.

29. The lateral flow test strip of claim 27 wherein the strip is configured such that the elution reagent combined with the sample is added to the sample application region of the strip.

30. The lateral flow test strip of claim 27 wherein the elution reagent raises the pH of the sample solution to provide neutralization.

31. The lateral flow test strip of claim 27 wherein the elution reagent dilutes the sample to provide neutralization.

32. The lateral flow test strip of claim 27 wherein the elution reagent comprises a buffer.

33. A lateral flow test strip for detecting analyte levels in a sample comprising:
a sample application region;
a decomplexation region comprising a heated region which provides heat to dissociate analyte-antibody complexes in the sample;
a conjugate region comprising a detection antibody that selectively associates with the analyte;
a flow region; and
a test line comprising immobilized test antibody.

34. The lateral flow test strip of claim 33 further comprising an elution reagent application region on the strip upstream of the sample application region.

35. The lateral flow test strip of claim 33 wherein the strip is configured such that the elution reagent combined with the sample is added to the sample application portion of the strip.

36. The lateral flow test strip of claim 33 wherein the heated region comprises a reagent that release heat when the elution reagent comes into contact with the region.

37. The lateral flow test strip of claim 36 wherein the reagent that releases heat comprises calcium oxide.

38. The lateral flow test strip of claim 33 wherein the heated region is heated by an infrared source.

39. The lateral flow test strip of claim 33 wherein the heated region is heated by an electric heater.

40. The lateral flow test strip of claim 39 wherein the electric heater is powered by a battery.

41. A method for detecting an analyte, which analyte may comprise analyte-antibody complexes, in a sample comprising:
providing a test strip comprising;
a sample application region;
a decomplexation region for dissociating analyte-antibody complexes in the sample;
a conjugate region comprising a detection antibody that selectively associates with the analyte;
a flow region; and
a test line comprising immobilized test antibody, and
applying a sample to sample application region;
whereby the sample flows down the strip such that when the sample is in the decomplexation region at least some of the analyte-antibody complex is dissociated, and
whereby the sample passes through the test line whereby the presence of analyte is detected by complexation with the immobilized test antibody.

42. The method of claim 41 wherein the test strip further comprises a neutralization region downstream of the decomplexation region, and whereby the neutralization region provides a neutralizing agent.

43. The method of claim 41 wherein the test strip further comprises an elution reagent application region on the strip upstream of the sample application region.

44. The method of claim 41 wherein after the sample is applied, elution reagent is added to the elution reagent application region to facilitate flow.

45. The method of claim 41 wherein the sample is combined with elution reagent which is added to the sample application region of the strip.

46. The method of claim 41 wherein the method provides for measuring the level of analyte in the sample.

47. The method of claim 41 wherein the analyte comprises p24 analyte.

48. The method of claim 47 wherein the level of p24 analyte in the sample is measured.

49. The method of claim 41 wherein the decomplexation reagent comprises an acidification reagent that lowers the pH of the sample as the sample as it passes through the decomplexation region.

50. The method of claim 42 wherein the decomplexation reagent comprises an acidification reagent that lowers the pH of the sample as the sample as it passes through the decomplexation region, and wherein the neutralizing reagent comprises a base that raises the pH of the sample as it passes through the neutralization region.

51. The method of claim 50 wherein the acidification reagent brings the pH of the sample to less than about 5.

52. The method of claim 50 wherein the acidification reagent brings the pH of the sample to less than about 4.

53. The method of claim 50 wherein the acidification reagent brings the pH of the sample to less than about 3.

54. The method of claim 50 wherein the acidification reagent comprises citric acid, glycine-HCl, or tartaric acid.

55. The method of claim 50 wherein the acidification reagent comprises a polymeric cation exchanger in the acid form.

56. The method of claim 50 wherein the acidification reagent comprises a carboxylic acid, a sulfonic acid, a phosphoric acid or a phosphonic acid.

57. The method of claim 41 or 42 wherein the decomplexation reagent comprises a detergent.

58. The method of claim 41 or 42 wherein the decomplexation region raises the salt concentration in the sample for decomplexation.

59. The method of claim 58 wherein the decomplexation reagent comprises lithium chloride, magnesium chloride, or sodium thiocyanate.

60. The method of claim 41 or 42 wherein the decomplexation region provides an organic solvent into the sample for decomplexation.

61. The method of claim 60 wherein the organic solvent comprises ethylene glycol.

62. The method of claim 41 wherein the decomplexation region is a region that is heated.

63. The method of claim 62 wherein the heating is provided by a compound that gives off heat when it comes in contact with the elution reagent.

64. The method of claim 62 wherein the heating is provided by an electric heater.

65. The method of any of claim 41-64 wherein the detection antibody comprises a fluorescent label.

66. The method of claim 41-65 wherein the decomplexation region and the sample application region are coextensive

67. The method of claim 42 wherein the neutralization region is coextensive with the conjugate region.

68. A method of determining the level of p24 in a patient in order to manage HIV therapy comprising;
obtaining a sample from a patient;
applying the sample to a lateral flow test strip of claim 1-31;
determining the level of p24 in the sample;
using the determined level of p24 to manage the care of the HIV patient.

69. The method of claim 68 wherein the sample comprises whole blood from the patient.

70. The method of claim 68 wherein the sample comprises serum from the patient.

71. The method of claim 68 wherein the p24 level is determined using a fluorescently labeled detection antibody.

72. A dual flow test strip for detecting analyte levels in a sample in which the analyte may be complexed comprising:
a sample application region;
wherein the strip comprises, downstream of the sample application region, a first lane and a second lane, wherein the first lane comprises;
a decomplexation region for dissociating analyte-antibody complexes in the sample;
a first lane flow region; and
a first lane test line comprising immobilized test antibody;
wherein the second lane does not have a decomplexation region and comprises;
a second lane flow region; and
a second lane test line comprising immobilized test antibody.

73. The dual flow test strip of claim 72 further comprising an elution reagent application region on the strip upstream of the sample application region.

74. The dual flow test strip of claim 72 wherein the first lane and the second lane each further comprise a conjugate region comprising a detection antibody that selectively associates with the analyte before the test line.

75. The dual flow test strip of claim 72 wherein the strip is configured such that the elution reagent combined with the sample is added to the strip along with the sample.

76. The dual flow test strip of claim 72 wherein the detection antibody comprises a fluorescent label.

77. A method for measuring both decomplexed and complexed analyte levels in a sample comprising:
adding a sample containing analyte that may be complexed to dual flow test strip, the test strip comprising:
a sample application region;
wherein the strip comprises, after the sample application region, a first lane and a second lane, wherein the first lane comprises;
a decomplexation region for dissociating analyte-antibody complexes in the sample;
a first lane conjugate region comprising a detection antibody that selectively associates with the analyte
a first lane flow region; and
a first lane test line comprising immobilized test antibody
wherein the second lane does not have a decomplexation region and comprises;
a second lane conjugate region comprising a detection antibody that selectively associates with the analyte
a second lane flow region; and
a second lane test line comprising immobilized test antibody; and
measuring signal corresponding to the detection antibody at both the first lane test line and at the second lane test line to determine both decomplexed and complexed analyte levels in a sample.

78. The method of claim 77 wherein the strip is configured such that an elution reagent combined with the sample is added to the strip at the sample addition region.

79. The method of claim 77 wherein after the sample is applied, an elution reagent is added to an elution reagent application region to facilitate flow.

80. The method of claim 77 wherein the measured signal comprises a fluorescent signal.

81. A lateral flow test strip for detecting analyte levels in a sample comprising:
a sample application region;
a conjugate region comprising a detection antibody that selectively associates with the analyte;
a flow region; and
a test line comprising immobilized test antibody, wherein the test line is narrower than the width of the test strip, and its length to width ratio y to x is greater than 2:1, where the length y is parallel to the direction of flow.

82. The lateral flow test strip of claim 81 further comprising an elution reagent application region upstream of the sample application region.

83. The lateral flow test strip of claim 81 wherein the strip is configured such that an elution reagent combined with the sample is added to the sample application region of the strip.

84. The lateral flow test strip of claim 81 wherein the length to width ratio y to x of the test strip is greater than 3:1.

85. The lateral flow test strip of claim 81 wherein the length to width ratio y to x of the test strip is greater than 5:1.

86. The lateral flow test strip of claim 81 wherein the length to width ratio y to x of the test strip is greater than 10:1.

87. A lateral flow test strip for determining analyte levels in a sample comprising:
a sample application region;
a conjugate region comprising a detection antibody that selectively associates with the analyte;
a flow region; and
a test line comprising immobilized test antibody, wherein the test line comprises a plurality of test regions, each of the regions comprising the same test antibody.

88. The lateral flow test strip of claim 87 further comprising an elution reagent application region on the strip upstream of the sample application region.

89. The lateral flow test strip of claim 87 wherein the strip is configured such that the elution reagent combined with the sample is added to the sample application region of the strip.

90. The lateral flow test strip of claim 87 wherein the test line comprises from about 4 to about 100 test regions.

91. The lateral flow test strip of claim 90 wherein the test line comprises an array of test regions.

92. The lateral flow test strip of claim 91 wherein the array of test regions is an array of n by p regions where n and p are independently 2, 3, 4, 5, 6, 7, 8, 9, or 10.

93. A lateral flow test strip for determining analyte levels in a sample comprising:
a sample application region;
a conjugate region comprising a detection antibody that selectively associates with the analyte;
a flow region; and
a test line comprising immobilized test antibody, wherein the test line comprises at least two portions, a high sensitivity portion having a length to width ratio y to x that is less than 2:1, and a high dynamic range portion having a length to width ratio y to x that is greater than 2:1, wherein the length y is in the direction of flow.

94. The lateral flow test strip of claim 93 further comprising an elution reagent application region on the strip upstream of the sample application region.

95. The lateral flow test strip of claim 93 wherein the strip is configured such that an elution reagent combined with the sample is added to the sample application region of the strip.

96. The lateral flow test strip of claim 93 wherein the high sensitivity portion has a length to width ratio y to x that is less than 3:1, and a high dynamic range portion having a length to width ratio y to x that is greater than 3:1.

97. The lateral flow test strip of claim 93 wherein the high sensitivity portion has a length to width ratio y to x that is less than 5:1, and a high dynamic range portion having a length to width ratio y to x that is greater than 5:1.

98. The lateral flow assay of claim 93 wherein the detection antibody comprises a fluorescent label.

99. A lateral flow assay method for detecting analyte levels in a sample with a reduced Prozone effect comprising:
providing a lateral flow assay test strip comprising:
a sample application region;
a flow region; and
a test line comprising immobilized test antibody;
adding a solution comprising the sample to the strip, whereby the sample flows up the strip toward the test line;
subsequently adding a solution comprising a detection antibody that selectively associates with the analyte; whereby the sample reaches the test line prior to the arrival of the detection antibody.

100. The lateral flow assay method of claim 99 wherein an elution reagent is applied after the addition of sample in order to facilitate flow.

101. A lateral flow assay test strip for detecting analyte levels in a sample providing for a reduced Prozone effect comprising:
an elution reagent addition region;
a portion of the strip downstream of the elution reagent addition region having a sample lane and a conjugate lane,
wherein the sample lane comprises a sample application region, and
the conjugate lane comprises a conjugate region, comprising a detection antibody that selectively associates with the analyte;
a flow region, and
a test line comprising immobilized test antibody, the test strip configured such that sample is added to the sample addition region, and elution reagent is added to the elution reagent addition region, whereby the elution reagent flows down both the sample lane and the conjugate lane, and the rate of travel down the strip for the detection antibody in the conjugate lane is slower than the rate of travel down the strip for the sample in the sample lane, whereby the sample reaches the test strip before the detection antibody reaches the test strip.

102. The lateral flow assay test strip of claim 101 wherein the conjugate lane comprises an altered fluid flow path.

103. The lateral flow assay test strip of claim 101 wherein the conjugate lane comprises a serpentine flow path.

104. The lateral flow assay test strip of claim 102 wherein the altered fluid flow path is produced by interdigitated hydrophobic barrier lines.

105. A lateral flow test strip for detecting analyte levels in a sample having improved sensitivity comprising:
a sample application region;
a decomplexation region comprising a decomplexation reagent for dissociating analyte-antibody complexes in the sample;
a conjugate region comprising a detection antibody that selectively associates with the analyte;
a flow region; and
a test line comprising immobilized test antibody, wherein the width of the flow path in the lateral flow test strip at the test line is 80% or less of the width of the flow path at the sample addition region.

106. The lateral flow assay test strip of claim 105 wherein the width of the flow path at the test line is 50% of the width of the flow path in the sample addition region.

107. The lateral flow assay test strip of claim 105 wherein the width of the flow path at the test line is 20% of the width of the flow path in the sample addition region.

108. A portable fluorescent reader for detecting analyte levels in a sample, comprising:
an illumination source providing excitation light;
illumination optics for directing the illumination light to a lateral flow test strip;
a region for holding a lateral flow test strip of any of the above claims, the lateral flow assay strip comprising a test line;
light collection optics for directing light emitted from the test line on the lateral flow test strip to a detector; and
a detector comprising a camera.

109. The portable fluorescent reader of claim 108 wherein the detector comprises a cell phone.

110. The portable fluorescent reader of claim 108 further comprising a processor for analyzing data from the camera.

111. The portable fluorescent reader of claim 108 wherein the illumination light comprises an LED.
